(19) United States
(12) Patent Application Publication
(10) Pub. No.: US 2026/0009716 A1
(43) Pub. Date: Jan. 8, 2026

(54) QUANTITATIVE MEASUREMENT OF MOLECULES USING SINGLE MOLECULE FLUORESCENCE MICROSCOPY

(71) Applicants: Oxford Nanoimaging Inc., La Jolla, CA (US); Oxford Nanoimaging Limited, Oxford, Oxfordshire (GB)

(72) Inventors: James Rhodes, Oxford, Oxfordshire (GB); Bo Jing, Oxford, Oxfordshire (GB); James Hannes Felce, Oxford, Oxfordshire (GB)

(73) Assignees: Oxford Nanoimaging Inc., La Jolla, CA (US); Oxford Nanoimaging Limited, Oxford, Oxfordshire (GB)

(21) Appl. No.: 18/993,524

(22) PCT Filed: Jul. 12, 2023
(86) PCT No.: PCT/EP2023/069380

§ 371 Date: Jan. 10, 2025

Related U.S. Application Data
(60) Provisional application No. 63/388,337, filed on Jul. 12, 2022.

(51) Int. Cl. G01N 15/1434; G01N 15/01; G01N 33/58

(52) U.S. Cl. CPC G01N 15/1434; G01N 15/01; G01N 33/582

(57) ABSTRACT

The present application discloses single molecule fluorescence methods for quantitating the presence of target molecules in a biological sample. The method involves labelling target molecules of the biological sample with a probe, the probe comprising at least one photoactivatable fluorophore and then imaging the target molecules. The imaging involves carrying out multiple imaging cycles, each imaging cycle having an activation step, an excitation step and a photobleaching step. The application also discloses probes suitable for use in quantitative single molecule fluorescence microscopy assays, as well as diagnostic methods based on quantitating the presence of target molecules.

This application claims priority from U.S. provisional patent application No. 63/388,337 filed 12 Jul. 2022, the contents and elements of which are herein incorporated by reference for all purposes.

### FIELD OF THE INVENTION

The present invention relates to methods and reagents for quantifying the presence of biomarkers in a sample, and in particular to methods and reagents for quantifying the copy number of biomarkers in or on a cell.

### BACKGROUND

There is great value in being able to identify not only the presence, but also the abundance of molecules in a sample. At a cellular level, the copy number of particular proteins, for example, can be important for identifying the severity of disease states, or the receptiveness of a patient to a particular treatment.

A common approach to quantifying the prevalence of biomarkers is to carry out an enzyme-linked immunosorbent assay (ELISA). Whilst ELISA provides reasonable accuracy, it can only be used to quantify the presence of the biomarker in the sample as a whole. The technique cannot be used to measure and quantify biomarkers at a single cell level (e.g. to identify heterogeneity between cells and/or different cell populations), nor to provide information about the spatial distribution of the biomarker within the cell.

To quantify biomarker prevalence at a single cell level, various fluorescence-based techniques have been used. For example, it is known to identify the prevalence of specific biomarkers in cells by fluorescently labelling the biomarkers, and then observing the overall fluorescence intensity detected from that cell using widefield microscopy, e.g. using a flow cytometer. However, these methodologies are relatively insensitive, giving only a broad estimate of the amount of biomarkers expressed, and being unsuitable for use at low copy numbers. In addition, the techniques do not allow accurate information to be obtained about the location of the biomarkers relative to the cell.

It is also known to obtain estimates of biomarker numbers using fluorescence correlation spectroscopy (FCS) or fluorescence fluctuation spectroscopy (FFS). In this technique, fluctuations in the detected fluorescence intensity from a region (typically a confocal volume) are used to obtain the concentration of biomarkers in the cell. However, this methodology becomes complicated in situations where the distribution of biomarkers across or within the cell is inhomogeneous. In the case of confocal methodologies, this approach does not provide information about the specific locations of the biomarkers outside of the small confocal volume.

Another common approach has been to observe step-wise photobleaching of fluorescently labelled molecules within a sample to identify copy number. However, this methodology relies on being able to resolve individual downward steps in the detected signal intensity as individual fluorophores are photobleached, and hence is only suitable where the number of fluorophores being detected is relatively small. For this reason, the method has typically been used to determine the stoichiometry of individually-observed protein complexes, instead of to determine copy numbers at a cellular level.

More recently, super-resolution fluorescence microscopy techniques which rely on identification of individual molecules within a sample have also been used to identify the prevalence of specific biomarkers. For example, in photoactivated localisation microscopy (PALM) a sparse subset of fluorophores is photoactivated from a dark state to an active state using a light source, the activated fluorophores are made to fluoresce using a readout laser beam until the fluorophore photobleaches, and the position of each fluorophore is identified. However, whilst this technique can allow exquisite accuracy in identifying the position of fluorophores, existing approaches can present challenges in terms of achieving accurate quantitative information about biomarker number and distribution compared to ground-truth, and the technique can be relatively slow to implement.

The present invention has been devised in light of the above considerations.

### SUMMARY OF THE INVENTION

At its broadest, the present invention provides improved single-molecule fluorescence-based methods for quantitating the number of target molecules in a sample.

In particular, the present inventors have identified that previously-applied single molecule methods for counting biomolecules are prone to significant error in terms of either overcounting or undercounting the true number (“ground truth”).

For example, photoblinking (in which an activated fluorophore temporarily enters a “dark” non-fluorescent state before re-entering its activated fluorescent state) can lead to inaccurate results when determining the number of those target molecules. These photoblinking events can cause a single fluorophore to be counted as multiple separate target molecules. Whilst it is known to try to compensate for this photoblinking by allowing a fluorescent spot to have a certain number of “dark” or “gap” frames in a video, this type of compensation is not perfect, and carries the risk of undercounting in situations where closely-spaced fluorophores are mistakenly identified as a photoblinking event.

In addition, the inventors have recognised that the fluorescent labelling strategy used in single molecule methods can have an important impact on accurately determining the numbers of molecules in a sample.

In view of the above considerations, in a first aspect, the present invention provides a method of carrying out single-molecule imaging of a target molecule in a biological sample, the method comprising:
a) labelling target molecules of the biological sample with a probe, the probe comprising at least one photoactivatable fluorophore;
b) imaging the (labelled) target molecules through carrying out multiple imaging cycles, each imaging cycle having:
   i. an activation step, comprising delivering activation light from an activation light source to photoactivate a subset of the photoactivatable fluorophores into photoactivated fluorophores;
   ii. an excitation step, comprising illuminating the photoactivated fluorophores with an excitation light source, and measuring the detected fluorescence to identify the individual photoactivated fluorophores; and optionally
   iii. a photobleaching step, comprising photobleaching the individual photoactivated fluorophores by illuminating the sample at a higher intensity than that used for the excitation step.

Preferably, the method is used as a method of quantitating/quantifying the presence of the target molecule. In other words, the method may be used as a method of counting the number of target molecules in a biological sample. For the avoidance of doubt the terms “quantitating” and “quantifying” are used synonymously in this application.

Preferably, the biological sample comprises cells. In such instances, the method may be a method of quantitating/quantifying the presence of a target molecule on individual cells of a biological sample.

In conventional PALM the activation step and excitation step are carried out at the same time. For example, a UV laser is applied continuously to randomly photoactivate a subset of the photoactivatable fluorophores, and the excitation light source is likewise applied continuously in order to image fluorophores as they become activated. However, in instances where imaging identifies two putative fluorophores located close to one another separated by one or more gap frames, this conventional methodology means it is not possible to definitively distinguish between two signals arising from photoblinking of a single fluorophore or activation of two separate fluorophores. In contrast, in the method of the invention, photoactivating the fluorophores using a pulse of activation light means that any gaps in the fluorescence during the excitation step can be attributed definitively to photoblinking, thus avoiding ambiguity about the number of fluorophores contributing to the signal.

Suitably, all photoactivated fluorophores in a given imaging cycle are photobleached before continuing to the next imaging cycle.

In conventional PALM microscopy, involving the continuous provision of both activation and excitation light, photobleaching is generally delayed as much as possible in order to boost the number of photons detected from a given fluorophore, in order to maximise location accuracy. However, the present inventors have recognised that actively promoting more rapid photobleaching in the context of the method of the invention can help to increase the speed at which molecule counting data can be obtained. Thus, preferably, each imaging cycle includes a photobleaching step in which photobleaching of the fluorophores is actively promoted.

The present invention encompasses a number of ways to promote photobleaching during the photobleaching step.

Preferably, the photobleaching step involves illuminating the sample/photoactivated fluorophores at a higher intensity than that used for the excitation step. For example, in preferable implementations the photobleaching step involves increasing the intensity of the excitation light source after the excitation step in order to promote photobleaching, for example, by providing a pulse of higher intensity excitation light. In other words, the method may involve illumination using the excitation light source at a lower intensity during the excitation step, and illumination using the excitation light source at a higher intensity during the photobleaching step.

As an example, the illumination intensity during the photobleaching step may be at least 50%, at least 100% higher (i.e. double), at least 150% higher, at least 200% higher (i.e. triple), at least 300% (i.e. four times), at least 400% (i.e. at least five times) or at least 500% (i.e. at least six times) higher (preferably at least 100% higher—i.e. double) than the illumination intensity used during the excitation step. The illumination intensity may be measured using an optical power meter, in a manner known to those skilled in the art. The illumination intensity may be increased, for example, by increasing the power supply to the light source, by more tightly focussing the illumination intensity (e.g. by periodically moving a focussing lens or mirror into the beam path) and/or by altering the degree of attenuation of the excitation light (e.g. using optical density filters).

To aid photobleaching, the method may comprise treating the biological sample with a chemical which promotes fluorophore photobleaching. For example, when the biological sample is immersed in a solution (e.g. an aqueous solution), the solution may contain a chemical which covalently reacts with the fluorophore in its excited state to create a non-fluorescent species. Suitable chemicals may be an oxidising agent, such as hydrogen peroxide or sodium hypochlorite.

### Probes

Optionally, the probe is a photoactivatable fluorophore directly attached to the target molecule. Preferably, the probe is a capture molecule having specificity for the target molecule, where the capture molecule bears one or more photoactivatable fluorophores (alternatively referred to herein as a “fluorescently-labelled capture molecule”). Suitable fluorescently-labelled capture molecules include, for example, an antibody or an antibody fragment, aptamer, nucleic acid, polypeptide, or a purified or synthetic ligand. Examples of suitable antibody fragments may include, for example, a F(ab′)2, F(ab)2, Fab′, Fab, variable fragment (Fv), single chain variable fragment (scFv), diabodies, linear antibodies, single-chain antibody molecules, and multispecific antibodies formed from antibody fragments. Preferably, the antibody is a monoclonal antibody, or an antibody fragment derived therefrom.

The photoactivatable fluorophore may be, for example, a fluorescent protein (“FP”) or an organic dye.

Examples of photoactivatable fluorescent proteins include:
PA-GFP [fluorophore excitation 504 nm, emission 517 nm (green), photoactivated with UV-Violet (405 nm) for high emission or blue (488 nm) laser for low emission],
PA-TagRFP [fluorophore excitation 562 nm, emission 595 nm (red), photoactivated with UV-Violet (405 nm)]
PA-mCherry1 [fluorophore excitation 570 nm, emission 596 nm (red), photoactivated with UV-Violet (405 nm)]
PA-mKate2 [fluorophore excitation 586 nm, emission 628 nm (far-red), can be photoactivated with UV-Violet (405 nm)].

Optionally, the photoactivatable fluorescent protein is conjugated to the target molecule. However, this methodology is not preferred in instances where accurate counts of endogenous target molecules are required, for several reasons. Firstly, modifying the DNA of the cell so that the gene encoding the target molecule is replaced with the gene encoding the target molecule FP conjugate is not straightforward, and there is no guarantee that the expression level of the conjugate will be equivalent to that of the target molecule. Secondly, such modifications cannot be carried out directly on samples from patients, but instead requires modification and replication of cells. Thirdly, the methodology can be used in situations where the target molecule is a protein, but is not suited to situations where the target molecule is a non-protein—e.g., a lipid, or nucleic acid.

For these reasons, in instances where photoactivatable fluorescent proteins are used, it is preferred that these are conjugated to a capture molecule. In this way, the capture molecule can be used to probe endogenously expressed target molecules.

Preferably, the fluorophores comprise or consist of photoactivatable organic dyes. Examples of photoactivatable organic dyes include, for example:
PA Janelia Fluor® 549, SE [fluorophore excitation 549 nm, emission 571 nm, photoactivated with UV-Violet (405 nm), available from Tocris Bioscience, Bristol, UK]
PA Janelia Fluor® 646, SE [fluorophore excitation 646 nm, emission 664 nm, photoactivated with UV-Violet (405 nm), available from Tocris Bioscience, Bristol, UK]
Abberior CAGE 500 [fluorophore excitation 511 nm, emission 525 nm, photoactivated with UV-Violet (405 nm), available from Abberior GmbH, Göttingen, Germany]
Abberior CAGE 532 [fluorophore excitation 552 nm, emission 574 nm, photoactivated with UV-Violet (405 nm), available from Abberior GmbH, Göttingen, Germany]
Abberior CAGE 552 [fluorophore excitation 595 nm, emission 615 nm (red), photoactivated with UV-Violet (405 nm), available from Abberior GmbH, Göttingen, Germany]
Abberior CAGE 590 [fluorophore excitation 595 nm, emission 615 nm (red), photoactivated with UV-Violet (405 nm), available from Abberior GmbH, Göttingen, Germany]
Abberior CAGE 635 [fluorophore excitation 630 nm, emission 647 nm, photoactivated with UV-Violet (405 nm), available from Abberior GmbH, Göttingen, Germany]

Most preferably, the probe is a capture molecule labelled with one or more photoactivatable organic dyes.

The fluorophores may be bonded directly to the target molecule or capture molecule via a tag, for example a protein tag, peptide tag or oligonucleotide tag.

Examples of suitable protein tags include, for example, HaloTag, SNAP-tag, CLIP-tag. Details of methodologies for attaching photoactivatable Janelia Fluor® dyes to such tags can be found in, for example, Grimm, J. B. et al “Synthesis of Janelia Fluor HaloTag and SNAP-Tag Ligands and Their Use in Cellular Imaging Experiments” in Chapter 15 of Super-Resolution Microscopy: Methods and Protocols, Methods in Molecular Biology, vol. 1663, published 2017.

The oligonucleotide tag may be used as part of a hybridisation probe. In other words, an oligonucleotide tag may be attached to the target molecule or capture molecule, and then hybridised with a complementary oligonucleotide bearing a photoactivatable fluorophore. Oligonucleotide tags may be attached using methodologies known in the prior art, for example, according to the methods taught in Wiener et al.

Preferably the degree of labelling (DOL) of the target molecule with the photoactivatable fluorophore is between 1.0 and 2.0, preferably between 1.0 and 1.5, more preferably between 1.0 to 1.2, most preferably 1.0. In instances where the probe is a capture molecule, the degree of labelling of the capture molecule with the photoactivatable fluorophore may be between 1.0 and 2.0, preferably between 1.0 and 1.5, more preferably between 1.0 to 1.2, most preferably 1.0. By “degree of labelling” we mean the number of fluorophores associated with each target molecule. Typically, this will be measured across the sample of target molecules as a whole, hence the reported value may be a non-integer, to account for variation in labelling across the sample.

Preferably, at least 80% of target molecules bear only one photoactivatable fluorophore (i.e. the degree of labelling is 1), more preferably at least 90% of target molecules, more preferably at least 95% of target molecules, more preferably at least 98% of target molecules, most preferably all target molecules.

In instances where the probe is a capture molecule, it is preferred that at least 80% of the capture molecules bear only one photoactivatable fluorophore (i.e. the degree of labelling is 1), more preferably at least 90% of capture molecules, more preferably at least 95% of capture molecules, more preferably at least 98% of capture molecules, most preferably all capture molecules.

The degree of labelling may be determined based on knowledge of the labelling methodology. For example, in instances where the probe necessarily bears a known number of fluorophores (e.g. through introduction of an FP moiety) the degree of labelling corresponds to this known number of fluorophores.

In instances where the degree of labelling cannot be accurately determined based on knowledge of the labelling methodology, the degree of labelling may be determined using UV-Vis spectroscopic methodologies. This may be achieved, for example, by:
(1) removing excess photoactivatable probe from the probe, e.g. by dialysis or gel filtration;
(2) photoactivating the photoactivatable fluorophores by applying light at a suitable wavelength to achieve activation of all (or at least substantially all) of the photoactivatable fluorophores;
(3) measuring the absorbance of the photoactivated fluorescently-labelled probe at 280 nm using a spectrophotometer cuvette having a 1 cm path length;
(4) measuring the absorbance of the photoactivated fluorescently-labelled probe at the maximum absorbance wavelength of the photoactivated fluorophores used;
(5) calculating the molarity of the probe according to the formula:

$$Protein concentraion ( M ) = \frac{{A}_{280} - ( {A}_{max} \times CF ) \times dilution factor}{ε}$$

where ε is the molar extinction coefficient of the molecule to which the photoactivated fluorophores are attached; Aₘₐₓ is the absorbance of a solution of the photoactivatable fluorophores when measured at the wavelength maximum after photoactivation of the fluorophores; CF is a correction factor to adjust for the amount of absorbance at 280 nm caused by the activated fluorophore (information available from the fluorophore manufacturer); and the “Dilution factor” is the extent to which the probe was diluted for the absorbance measurement;
(6) calculating the degree of labelling according to the formula:

$$DOL = \frac{{A}_{max} of the labelled probe \times dilution factor}{{ε}_{dye} \times protein concentration}$$

where ε_{dye} is the molar extinction coefficient of the fluorescent dye; and
(7) optionally, repeating steps (2)-(6) at using different conditions for step (2) to ensure that maximum photoactivation of the photoactivatable fluorophores has been achieved. For example, the method may be repeated multiple times ramping up the photoactivation time and/or intensity at each repetition.

Typically, steps (2)-(4) are carried out on the same sample in the same spectrophotometer. For example, the sample may be placed in a cuvette, initially illuminated with light at a wavelength to achieve photoactivation (e.g. at a wavelength of 405 nm) and then illuminated with light to carry out the absorbance measurements.

Advantageously, labelling with a known number of fluorophores allows accurate quantification of the target molecule. In particular, if the DOL of each target molecule is known to be a specific number, an accurate measure of the total number of target molecules can be obtained by dividing the total number of detected fluorophores by the DOL. Furthermore, the inventors have recognised that minimising the DOL can simplify the quantification procedure, and also increase the speed of experiments. In particular, the inventors have recognised that it is particularly advantageous when DOL=1. For example, if there are N fluorescently-labelled target molecules in a sample, and we must detect all fluorophores to accurately quantify N, then we must measure double the number of fluorophores when DOL=2 compared to when DOL=1. Furthermore, for a given activation light source power, the number of fluorophores activated during step (b)(i) when DOL=2 will be more than when DOL=1, so it may be necessary to reduce the activation rate (discussed below) as the degree of labelling increases, thereby further slowing data acquisition. As an additional advantage, reducing the number of fluorophores per probe can reduce or avoid photodynamic complications, in particular Förster resonance energy transfer (FRET) between fluorophores on the same probe.

In view of the above advantages, the present invention also provides specific fluorescent labelling strategies to give well-defined DOL of target molecules.

Thus, in some implementations the probe is an (primary) antibody labelled with a secondary antibody or antibody fragment, wherein the secondary antibody or antibody fragment bears a controlled number of fluorophores. The secondary antibody or antibody fragment is preferably a monovalent antibody fragment or variant. “Monovalent” in this context means an antibody fragment or variant comprising a single antigen-binding site. Examples of suitable monovalent antibody fragments or variants may include, for example, a Fab, Fab′, single chain variable fragment (scFv) or nanobody.

In preferred implementations the probe is an (primary) antibody labelled with a secondary nanobody, wherein the secondary nanobody bears a controlled number of fluorophores. As is known to those in the art, a secondary nanobody (alternatively referred to as a “nanobody”) is a single-domain antibody (sdAb).

Antibodies are an excellent way in which to label target molecules, in view of the huge repertoire of already-available strongly-binding antibodies, and the well-established processes for making new antibodies. Typically, when used in fluorescence-based measurements, antibodies are labelled with amine-reactive fluorophores that react randomly with primary amines. However, antibodies will typically contain a range of amine sites available for labelling. Even where the relative stoichiometry of the amine-reactive fluorophore and antibody are controlled, the degree of labelling is not uniform, nor is the positioning of the fluorophores across the available labelling sites. Moreover, in situations where multiple fluorophores are present on a given antibody, complex photodynamics can arise through resonance energy transfer, causing further complications. Whilst in certain instances it is possible to engineer the antibody in order to control the number of available labelling sites, this closes off the possibility of using established antibodies, e.g. those available “off-the-shelf” from commercial suppliers.

To overcome these issues, the present inventors adopt an alternative labelling strategy. Specifically, the inventors use secondary antibodies having a controlled number of fluorophores, and attach these to a primary antibody which is used to bind the target molecules.

Preferably, the secondary antibodies are nanobodies. The use of nanobodies is advantageous because they can be expressed recombinantly, rather than being obtained by digesting whole antibodies. In addition, nanobodies are easy to engineer, small in size and strongly bind to their target antigen or target molecule.

Suitably, the nanobodies are monoclonal nanobodies.

Typically, control over the number of fluorophores is achieved by using a secondary antibody (e.g. a nanobody) having a controlled number of (ectopic) labelling sites, which are then reacted with fluorophores.

The controlled number of labelling sites may correspond to specific amino acid residues which can be labelled. In particular, the secondary antibody (e.g. nanobody) may have a controlled number of ectopic cysteine resides. The secondary antibody may have, for example, only 1 or 2, most preferably only 1, cysteine residue. Cysteine residues may be introduced through site-directed mutagenesis, using methods known in the art. The cysteine(s) may be labelled using a thiol-reactive fluorophore, such as a maleimide-functionalised fluorophore or iodoacetamide-functionalised fluorophore.

Alternatively, the controlled number of labelling sites may correspond to one or more tags, wherein the tags are labelled with a controlled number of fluorophores. The one or more tags may be protein tags, peptide tags or oligonucleotide tags, with protein tags being preferred. The protein tag may be, for example, a HaloTag (which covalently attaches to haloalkane substrates), a SNAP-tag (which covalently attaches to benzylguanine derivatives), or a CLIP-tag (covalently attaches to benzylcytosine derivatives). Protein tags may be introduced by standard engineering methods, known to those skilled in the art. Suitably, it is possible to precisely control the number of fluorophores on the binding partner of the protein tag. For example, PA Janelia Fluor® 549 and 646 are available in an N-hydroxysuccinimide (NHS) ester form, which can be reacted with haloalkanes, benzylguanine derivatives or benzylcytosine derivatives in a 1:1 ratio.

Suitably, the secondary antibodies (e.g. nanobodies) bind to an epitope on the constant region of the antibody. In this way, the secondary antibodies can be generally applied in the methods, regardless of the specific antibody used. Preferably, the secondary antibodies bind to an epitope in the Fc region, e.g. the C_{H}2 or C_{H}3 domain of an IgG antibody. Preferably, the secondary antibodies bind to an epitope in the final C_{H} domain of the Fc region, so as to minimise the chances of the attachment of the secondary antibodies disrupting the interaction between (primary) antibody and target molecule. Most preferably, the (primary) antibody is an IgG antibody, and the secondary antibodies bind to an epitope on the C_{H}3 domain, since this is furthest from the antigen binding site.

In instances where the secondary antibodies (e.g. nanobodies) bind to an epitope on the constant region of the (primary) antibody, there will be two possible binding sites per (primary) antibody. In such instances, if each secondary antibody is labelled with 1 fluorophore, the (primary) antibody itself will bear 2 fluorophores. In view of the advantages of having only 1 fluorophore per target molecule, the present inventors have devised strategies to ensure that each antibody probe has only a single fluorophore.

In a first strategy, the invention comprises a method of labelling a primary antibody with a single copy of a fluorophore F1, comprising:
I. a preparation step, comprising:
   providing a secondary antibody A having a first epitope tag, wherein the secondary antibody A has a single fluorophore F1 (the fluorophore may be added, e.g. via a Halotag, cysteine, or any other labelling system);
   providing a secondary antibody B with a second epitope tag, different from the first epitope tag, wherein the secondary antibody B lacks fluorophore F1;
wherein the secondary antibody A or secondary antibody B bind the same epitope on the primary antibody, or are cross-competing secondary antibodies (i.e. the binding of one secondary antibody to its epitope blocks binding of the other secondary to its epitope, for example due to overlap of the epitopes of the two secondary antibodies)
II. an incubation step, comprising incubating the primary antibody with secondary antibody A and secondary antibody B to provide an antibody-secondary antibody complex (e.g. an antibody-nanobody complex);
III. a purification step, comprising:
   performing a precipitation of the antibody-secondary antibody complex using one of the epitope tags, to obtain a first eluate containing the antibody-secondary antibody complex; and
   performing a precipitation of the first eluate using the other epitope tag to obtain a second eluate containing the antibody-secondary antibody complex;
wherein the second eluate comprises said primary antibody labelled with a single copy of fluorophore F1.

In preferred embodiments, the secondary antibody is a monovalent antibody fragment or variant. In particularly preferred embodiments, the secondary antibody is a nanobody.

Accordingly, in some embodiments, the method of labelling a primary antibody with a single copy of a fluorophore F1 comprises:
I. a preparation step, comprising:
   providing a secondary nanobody A having a first epitope tag, wherein the secondary nanobody A has a single fluorophore F1 (the fluorophore may be added, e.g. via a Halotag, cysteine, or any other labelling system);
   providing a secondary nanobody B with a second epitope tag, different from the first epitope tag, wherein the secondary nanobody B lacks fluorophore F1;
wherein the secondary nanobody A or secondary nanobody B bind the same epitope on the primary antibody, or are cross-competing nanobodies (i.e. the binding of one nanobody to its epitope blocks binding of the other nanobody to its epitope, for example due to overlap of the epitopes of the two nanobodies)
II. an incubation step, comprising incubating the primary antibody with secondary nanobody A and secondary antibody B to provide an antibody-nanobody complex;
III. a purification step, comprising:
   performing a precipitation of the antibody-nanobody complex using one of the epitope tags, to obtain a first eluate containing the antibody-nanobody complex; and
   performing a precipitation of the first eluate using the other epitope tag to obtain a second eluate containing the antibody-nanobody complex;
wherein the second eluate comprises said primary antibody labelled with a single copy of fluorophore F1.

Optionally, the secondary nanobody A and secondary nanobody B are separate molecules. In such instances, the step of incubating the primary antibody with the secondary nanobodies preferably comprises incubating the primary antibody with a mixture of the secondary nanobody A and secondary nanobody B. In such instances, the secondary nanobody A and secondary nanobody B are preferably mixed at a ratio of 1:1. In instances where the secondary nanobody A and the secondary nanobody B have different binding characteristics, the ratio may be adjusted to promote antibody-nanobody complexes bearing one copy of each secondary nanobody (“AB”).

When the secondary nanobody A and secondary nanobody B are separate molecules, incubating will result in a mixture of antibody-nanobody complexes bearing two copies of the same secondary nanobody (either “AA” or “BB”) and antibody-nanobody complexes bearing one copy of each secondary nanobody (“AB”). Advantageously, the co-immunoprecipitation step purifies the “AB” antibody-nanobody complexes.

Alternatively, the secondary nanobody A and secondary nanobody B may form part of a construct, in which the nanobodies are attached by a cleavable linker. In other words, the construct may have a formula:

$$( secondary nanobody A ) - linker - ( secondary nanobody B ) \qquad ( Formula 1 )$$

In such situations, the incubation step (II) comprises a further step of cleaving the cleavable linker. Such a cleavage step breaks apart any cross-linked primary antibodies that may have formed by the construct linking two primary antibodies, which might otherwise be pulled down during the precipitation step (III).

Suitably, the linker is a flexible linker. The linker is capable of allowing secondary nanobody A and secondary nanobody B to bind to their epitopes on a single primary antibody.

Advantageously, providing the secondary nanobody A and secondary nanobody B as part of a construct can lead to increased yield of AB antibody-nanobody complexes. For example, using a construct increases the amount of “AB” antibody-nanobody complex obtained, since intramolecular binding is generally faster and stronger than intermolecular binding. This reduces the amount of “unwanted” primary antibody labelled with either AA or BB.

The linker may be, for example, a photocleavable linker, a chemically cleavable linker, a thermally cleavable linker, or an enzymatically cleavable linker.

The construct may be, for example, a fusion protein in which the secondary nanobody A and secondary nanobody B are linked by a peptide linker. Advantageously, providing the construct in the form of a fusion protein can allow production of the nanobodies using a single procedure, instead of having to produce nanobody A and nanobody B separately.

Suitably, the peptide linker is an enzymatically cleavable linker. The peptide linker may comprise a peptide cleavage site, for example, a site which is cleavable by a protease such as TEV protease or 3C protease.

Suitably, the precipitation step involves co-immunoprecipitation. In such instances, the antibody-nanobody complex is precipitated by an antibody (or fragment or variant thereof) with specificity for the epitope tag.

Epitope tags suitable for use in the invention may be, for example, a peptide tag (such as an ALFA-tag, C-tag, E-tag, FLAG-tag, HA-tag, Lyc-tag, NE-tag, S-tag, Spot-tag, V4-tag, VSV tag or Xpress tag) or a protein tag (such as HaloTag, SNAP-tag, CLIP-tag).

Preferably, the tag on the secondary nanobody A enhances the photoactivation efficiency of the fluorophore F1. For example, the tag on secondary nanobody A may be a HaloTag and fluorophore F1 is attached to the HaloTag, since the HaloTag can boost the photoactivation efficiency of fluorophore F1. Methods for introducing protein tags is reported in Grimm et al, which also reports a boost in photoactivation efficiency when a photoactivatable fluorophore is attached to a HaloTag.

As well as allowing the labelling of antibodies with a single fluorophore, the methodologies above also have more general applicability to labelling strategies.

For example, in one variant secondary nanobody B bears an alternative functionality F2.

Functionality F2 may be, for example, a further fluorophore of a different kind to F1. Advantageously, providing an alternative fluorophore F2 can allow imaging in a second colour channel. In certain implementations, both F1 and F2 are photoactivatable fluorophores. In such situations, the method may involve carrying out the quantification method of the invention using both F1 and F2 to verify the quantity of a target molecule. Since many of the photoactivatable fluorophores above are activatable by the same activation light source (e.g. a light source operating at 405 nm), this means that it may be possible to carry out a quantification experiment in two colours simply by adding a further excitation light source.

Alternatively, functionality F2 may be a biotin moiety.

### Measurement Methods Based on Antibody-secondary Antibody Complexes

The present inventors believe that the labelling strategies set out above also constitute a new and non-obvious development.

Thus, in a second aspect the present invention provides a method of labelling a primary antibody with a single copy of a functional moiety F*, comprising:
I. a preparation step, comprising:
   providing a secondary antibody A (e.g. a secondary nanobody) A having a first epitope tag, wherein the secondary antibody A has a single copy of functional moiety F* (the functional moiety F* may be added, e.g. via a Halotag, cysteine, or any other labelling system);
   providing a secondary antibody B (e.g. a secondary nanobody B) with a second epitope tag, different from the first epitope tag, wherein the secondary antibody B lacks functional moiety F *;
wherein the secondary antibody A or secondary antibody B bind the same epitope on the primary antibody, or are cross-competing antibodies (i.e. the binding of one antibody to its epitope blocks binding of the other antibody to its epitope, for example due to overlap of the epitopes of the two antibodies)
II. an incubation step, comprising incubating the primary antibody with secondary antibody A and secondary antibody B to provide an antibody-secondary antibody complex;
III. a purification step, comprising:
   performing a precipitation of the antibody-secondary antibody complex using one of the epitope tags, to obtain a first eluate; and
   performing a precipitation of the antibody-secondary antibody complex using the other epitope tag to obtain a second eluate;
wherein the second eluate comprises said primary antibody labelled with a single copy of functional moiety F *. As noted above, in preferred implementations the secondary antibody A and secondary antibody B are both nanobodies.

In view of the aim of the present invention, in preferred embodiments the functional moiety F* is a fluorophore. In this broader aspect, the fluorophore can be of any type, and is not restricted solely to the photoactivatable fluorophores taught above.

A third aspect of the present invention also provides a fusion protein of formula:
(secondary nanobody A)-linker-(secondary nanobody B)
wherein secondary nanobody A, secondary nanobody B and the linker are as defined above.

A fourth aspect of the present invention provides a gene encoding the fusion protein of the third aspect.

A fifth aspect of the present invention provides a gene construct comprising the gene of the fourth aspect.

A sixth aspect of the present invention provides a cell comprising the gene construct of the fifth aspect.

A seventh aspect provides an antibody-nanobody complex comprising:
a primary antibody;
a secondary nanobody A, having a single fluorophore F1;
a secondary nanobody B, lacking a fluorophore F1;
wherein the secondary nanobody A and secondary nanobody B are bound to the primary antibody.

An eighth aspect of the present invention provides a composition comprising an antibody-nanobody complex, wherein at least 70% (preferably at least 80%, preferably at least 85%, preferably at least 90%, preferably at least 95%, preferably at least 98%, preferably essentially all) of the antibody-nanobody complexes present in the composition comprise:
a primary antibody;
a secondary nanobody A, having a single fluorophore F1;
a secondary nanobody B, lacking a fluorophore F1;
wherein the secondary nanobody A and secondary nanobody B are bound to the primary antibody.

Preferably, F1 is an organic dye.

Preferably, F1 is a photoactivatable fluorophore, most preferably a photoactivatable organic dye, such as PA Janelia Fluor® 549, PA Janelia Fluor® 646 or Abberior CAGE 590.

A ninth aspect of the present invention provides use of an antibody-nanobody complex of the seventh aspect or a composition of the eighth aspect in fluorescence microscopy. Preferably, the fluorescence microscopy is single-molecule fluorescence microscopy. Most preferably, F1 is a photoactivatable fluorophore, and the fluorescence microscopy is PALM.

A tenth aspect provides a method of carrying out PALM imaging of a biological sample, the method comprising:
a) labelling target molecules of the biological sample with a probe, the probe being an antibody-nanobody complex according to the seventh aspect of the invention, wherein F1 is a photoactivatable fluorophore;
b) imaging the biological sample by carrying out PALM microscopy of fluorophore F1.

An eleventh aspect provides a method of carrying out PALM imaging of a biological sample, the method comprising:
a) labelling target molecules of the biological sample with a composition according to the eighth aspect of the invention, wherein F1 is a photoactivatable fluorophore;
b) imaging the biological sample by carrying out PALM microscopy of fluorophore F1.

The present invention also extends to aspects corresponding to the seventh to tenth aspects, but with a different monovalent antibody fragment or variant, such as a Fab, Fab′, or single chain variable fragment (scFv).

### Imaging

The method of the invention may be carried out using a microscope configured to carry out single molecule localisation microscopy.

The microscope will typically comprise:
a stage, suitable for supporting an imaging substrate (e.g. a microscope slide) on which the biological sample is placed;
the activation light source, suitable for illuminating the biological sample when placed on the imaging substrate;
the excitation light source, suitable for illuminating the biological sample when placed on the imaging substrate;
an objective lens for collecting light emitted by the fluorophores; and
detection optics, comprising a detector for detecting light emitted by the fluorophores.

The microscope may be a wide-field illumination microscope, such as a widefield epifluorescence microscopy (in which the excitation light passes through the objective lens) or light sheet fluorescence microscopy (in which the excitation light source produces a sheet of light illuminated laterally at and parallel to the focal plane of the objective lens) or a Total Internal Reflection Fluorescence (TIRF) microscope.

Suitable detectors include a camera, for example a charge-coupled device (CCD), such as an electron-multiplying CCD (EMCCD), or a complementary metal-oxide semiconductor (CMOS) camera.

Each imaging cycle obtains images from a particular field of view. The field of view will be determined by, for example, the numerical aperture of the objective lens and the size of the detector. The field of view is generally chosen so as to maximise the region imaged whilst still allowing single molecule localisation.

Typically, the field of view imaged will constitute only a portion of the total amount of biological sample held on the imaging substrate. In such instances, the activation step preferably minimises photoactivation of fluorophores outside of the field of view. This helps to minimise the risk of unwanted photobleaching in regions outside of the field of view, which might otherwise lead to an underreporting of the number of target molecules in those regions. In other words, the activation light source may be configured such that at least 80% of its intensity, preferably at least 90% of its intensity is concentrated within the field of view. This may be achieved, for example, by using an epifluorescence or TIRF modality, and adjusting the beam size of the activation light source so that it only illuminates within the field of view. Adjustment of the beam size may be achieved, for example, using lenses to achieve a particular desired profile and/or by using apertures. Optionally, steps are taken to try to minimise variation in intensity of the activation light source within the field of view, for example through the use of apertures and/or beam homogenisers.

The size of the excitation light source profile relative to the field of view is typically less critical, since only activated fluorophores will be at risk of photobleaching. For example, the activation light source may be introduced in an epifluorescence or TIRF modality, and the excitation light source may be introduced in a light sheet modality. However, preferably, the activation light source and excitation light source are both introduced using an epifluorescence modality or TIRF, e.g. through the objective lens.

The activation light source and excitation light source operate at different wavelengths. This helps to decouple the activation step from the excitation step, so as to avoid (or at least minimise) the photoactivatable fluorophore becoming activated during the excitation step.

The specifics of the activation light source and excitation light source will be dependent on the characteristics of the fluorophores being used (guidance for which is given above in respect of specific photoactivatable fluorophores). However, typically, the activation light source will operate in the UV wavelength (for example, at a wavelength between 400-450 nm, such as around 405 nm) and the excitation light source will operate in the visible or near-infrared region (e.g. between 450-750 nm).

Suitably, the activation light source is turned off before the excitation step. In such implementations, the method generally involves multiple cycles of the illumination sequence: activation light source for the activation step, lower intensity excitation light source for the excitation step and (where used) higher intensity excitation light source for the photobleaching step. Advantageously, this method reduces the possibility of fluorophores being activated during the excitation step or photobleaching step, and photobleaching before a detectable signal is produced, which might otherwise lead to underreporting of the number of target molecules.

Preferably, the activation light source and excitation light source are lasers.

Optionally, the intensity of the activation light source is chosen so as to achieve a desired activation rate. By “activation rate” we mean the amount of activated fluorophores observed during the excitation step.

The activation rate may be chosen so as to minimise the incidence of closely-spaced activated fluorophores observed during the excitation step. This may be judged in a number of ways.

In a first implementation, the activation rate corresponds to the density of activated fluorophores across the whole field of view. This may be done, for example, by monitoring the total number of fluorophores detected in the field of view. Alternatively, this may be calculated by segmenting the field of view into an array of detection areas (of size A_{X}×A_{Y}), calculating the number of detected fluorophores in each detection area, and then calculating an average fluorophore density. This “whole field of view” approach works well in samples where the fluorophores are evenly spread across the field of view (for example, when dealing with target molecules in solution which have attached to the slide). However, the method is not so well suited to instances where the distribution of fluorophores across the field of view is inhomogeneous, for example, in instances where the target molecules are present on cells which are sparsely positioned across the field of view. In such instances, alternative methodologies may be more appropriate.

For example, in a second implementation, the activation rate corresponds to the local density of fluorophores. This may be calculated, for example, by assigning an area (of A_{X}×A_{Y}) around the central position of a detected fluorophore in each frame, and calculating the number of fluorophores which occur within that area in the same frame. In this way, a “local density” around the fluorophore is calculated. Calculating a “local density” in this way minimises artificially low calculated activation rates in samples where the target molecules are clustered across the field of view, e.g. in which the target molecules are associated with sparsely distributed cells. The “local density” may also be calculated using a rolling-ball algorithm.

In a third implementation, the activation rate corresponds to the nearest-neighbour distance between fluorophores. The nearest neighbour distance may be calculated based on the localisation positions calculated for the fluorophores. Again, as with the second implementation, this methodology minimises artificially low calculated activation rates in samples where target molecules are clustered across the field of view.

Optionally, the “local density” or nearest-neighbour distances may be calculated for only a subset of the detected fluorophores, to avoid excessive computing power being required to determine the activation rate. This can make it simpler for “on-the-fly” calculation of activation rate during the course of a measurement. This may be done, for example, by calculating the local density/nearest-neighbour distance for only a specific proportion of the detected fluorophores in each frame, for example, no more than 90% of the detected fluorophores, no more than 80% of the detected fluorophores, no more than 70% of the detected fluorophores or no more than 60% of the detected fluorophores. This proportion may be determined across the entire field of view. Alternatively, the subset may be calculated by segmenting the field of view into an array of areas, and selecting detected fluorophores in that area up to a certain threshold (to avoid oversampling in a specific area of the field of view). Preferably, however, the local density is calculated in respect of each detected fluorophore.

Generally, A_{X} and A_{Y} may be independently selected, and may be for example between 1 μm to 10 μm, 1 μm to 8 μm, 1 μm to 7 μm, 1 μm to 5 μm, 1 μm to 4 μm, or 1 μm to 3 μm. Preferably A_{X} and A_{Y} are the same. The area defined by A_{X} and A_{Y} may be a parallelogram, or may be a circle or oval.

Optionally, the activation rate is converted into an average activation rate. The activation light source may be configured so that the average activation rate falls between certain values. For example, the activation light source may be configured such that the activation rate is lower than a threshold Xᵤₚₚₑᵣ and optionally higher than a lower threshold X_{lower}. In the case of the activation rate being measured based on a nearest-neighbour measurement, X_{lower} may be, for example, 0.5 μm, 1 μm or 2 μm, and/or Xᵤₚₚₑᵣ may be, for example, 3 μm, 4 μm or 5 μm. In the case of the activation rate being based on density, X_{lower} may be 0.05 fluorophores per μm²; 0.1 fluorophores per μm²; 0.2 fluorophores per μm², 0.5 fluorophore per μm², or 1 fluorophores per μm², and Xᵤₚₚₑᵣ may be 10 fluorophores per μm²; 8 fluorophores per μm²; 6 fluorophores per μm², 5 fluorophore per μm², 4 fluorophores per μm², 3 fluorophores per μm², or 2 fluorophores per μm².

Alternatively, the activation light source may be configured to taking into account the population of activation rates, instead of an averaged value. For example, the activation light source may be configured such that incidences of the activation rate being less than a certain threshold X_{lower} are lower than Y %. Y % may be 10%, 5%, 4%, 3%, 2% or 1%. For example, incidences of the nearest neighbour distance between fluorophores being less than 1 μm may be less 5% of the total measured distances. The values for X_{lower} may be the same as those indicated above.

Similarly, the activation rate may be chosen to ensure that a sufficient density of fluorophores is observed during the excitation step, to achieve a suitable image acquisition rate. Again, this may be determined based on the different implementations taught above. For example, the incidences of the nearest neighbour distance between each observed fluorophore being above a certain threshold Xᵤₚₚₑᵣ may be less than Z %. Z % may be 10%, 5%, 4%, 3%, 2% or 1%. The values for Xᵤₚₚₑᵣ may be the same as those indicated above. For example, incidences of the nearest neighbour distance between fluorophores being more than 5 μm may be less 5% of the total measured distances.

Preferably, 90% of the nearest neighbour distances between each fluorophore may be, for example, between 0.5-5 μm, such as between 1-3 μm.

Preferably, the method incorporates a feedback loop to optimise the activation rate during the course of a measurement. In particular, the method may comprise monitoring the activation rate, and adjusting the characteristics of the activation light source (e.g. the intensity of the activation light source) in response to the measured activation rate. For example, the method may comprise increasing the power of the activation light source if the activation rate falls below a certain threshold, and decreasing the power of the activation light source if the activation rate exceeds a certain threshold. The threshold may be based on the field of view density, local density, or nearest neighbour distances, described above. For example, the method may involve determining the activation rate, and increasing the activation light source intensity if the activation rate is below a certain threshold (e.g. X_{lower}) and decreasing the activation light source intensity if the activation rate is above a certain threshold (e.g. Xᵤₚₚₑᵣ). For example, the method may involve determining the nearest neighbour distances between each fluorophore and increasing the activation light source intensity if 90% of the nearest neighbour distances are above Xᵤₚₚₑᵣ (e.g. 5, 4 or 3 μm) and/or decreasing the activation light source intensity if 10% or more of the nearest neighbour distances are below X_{lower} (e.g. 0.5 μm, 1 μm or 2 μm).

The intensity of the activation light source may be adjusted by, for example, increasing the power used to drive the activation light source and/or attenuating the beam, e.g. by using optical density filters.

Suitably, the feedback loop is computer-implemented.

This feedback loop can be used to maximise data acquisition rate, whilst still keeping the image sufficiently sparse to allow accurate localisation of individual fluorophores.

The inventors have recognised, in particular, that the activation rate may decrease during the course of an experiment as the number of non-photobleached photoactivatable fluorophores becomes smaller (as depicted in FIG. 11, discussed below). Thus, this feedback loop can be used to ensure, for example, that the data acquisition rate does not decrease significantly during the course of an experiment.

The feedback loop may incorporate defined operational boundaries for the activation light source. In particular, the feedback loop may be configured so that the power of the activation light source cannot exceed an upper boundary, to avoid damage to the sample or microscope.

In view of the advantages of this methodology, a further aspect of the invention provides a method of carrying out photoactivation localisation microscopy of photoactivatable fluorophores in a sample, comprising delivering activation light from an activation light source to photoactivate a subset of the photoactivatable fluorophores into photoactivated fluorophores, and imaging the photoactivated fluorophores, wherein the method comprises monitoring the activation rate of the photoactivatable fluorophores and adjusting the operation of the activation light such that the activation rate falls within a desired threshold.

Optionally, drift correction is carried out during imaging. For example, the method may involve cross-correlation based drift compensation. Alternatively, the drift correction may be based on entropy minimisation, following the approach of Cnossen, J. et al. Drift correction can be implemented using the Nanoimager available from Oxford Nanoimaging.

Suitably, the PALM is 2D-PALM.

In other implementations, the PALM is 3D PALM. In such techniques, astigmatism or defocusing is introduced into the imaging system, such that the shape of the detected fluorescence emission from a single molecule can be calibrated with the z position of the molecule. This may be achieved, for example, by introducing a cylindrical lens into the detection optics to create two different focal plans for the x and y directions, so that the shape of the spot detected varies according to the z position of the fluorophore. When fitted with a 2D elliptical Gaussian, as described below, the peak widths in the x and y directions can be related to the z position of the fluorescence emission. Details of such techniques can be found in, for example, Huang et al.

### Light Sequence

Suitably, the activation light is turned off during operation of the excitation light, and vice versa.

The activation light may be delivered as a pulse.

Likewise, photobleaching may be achieved as a pulse of higher intensity excitation light.

Thus, the imaging cycle may be carried out by implementing multiple repetitions of the sequence: activation light, lower intensity excitation light, higher intensity excitation light (suitably with no overlap between the activation light and excitation light).

The length of the excitation step may be, for example, no more than 10 seconds, no more than 8 second, no more than 6 seconds, no more than 5 seconds, no more than 2 seconds, no more than 1 second, no more than 0.8 seconds, no more than 0.7 seconds, no more than 0.6 seconds or no more than 0.5 seconds. In terms of camera frame rate, the excitation step may be carried out for no more than 200 frames, no more than 150 frames, no more than 100 frames, no more than 50 frames, no more than 40 frames, no more than 30 frames, no more than 20 frames, no more than 15 frames, no more than 10 frames, or no more than 5 frames.

The camera frame rate may be, for example, 30 frames per second, 33 frames per second, 50 frames per second, or 100 frames per second. Typical values for the excitation step may be, for example, 0.5 to 5 seconds, such as 1.5 to 4.5 seconds.

The activation step may be carried out, for example, for less than 200 ms, less than 100 ms, or less than 50 ms. The activation step may be carried out, for example, for between 10 to 200 ms, 10 to 100 ms, preferably 20 to 100 ms, preferably 30 to 90 ms.

The photobleaching step may be carried out, for example, for at least 200 ms, at least 500 ms, at least 1 second, or at least 2 seconds. The minimum time for the photobleaching step may be, for example, 50 ms, 100 ms, or 200 ms.

The overall time of each imaging cycle may be, for example, no more than 10 seconds, no more than 8 seconds, no more than 6 seconds, no more than 5 seconds, no more than 4 seconds, no more than 3 seconds, no more than 2 seconds, preferably no more than 1

### Fluorophore Localisation

Preferably the step of identifying the individual photoactivated fluorophores comprises identifying fluorophore position data for each fluorophore.

In single molecule imaging, the fluorescence from a fluorophore will appear as a spot, having an intensity distribution dictated by the point spread function of the system. To obtain position data, the point spread function is generally fitted to the signal from each fluorophore. The fitting may carried out with an elliptical point spread function. In one implementation, the signal from each fluorophore is fitted with a Gaussian function (approximating an Airy disk), for example a two-dimensional Gaussian function of the general form:

$$f ( x , y ) = A exp ( - ( \frac{{( x - {x}_{o} )}^{2}}{2 {σ}_{X}^{2}} + \frac{{( y - {y}_{o} )}^{2}}{2 {σ}_{Y}^{2}} ) )$$

where A is the peak height, x₀ and y₀ are the peak centres, x and y are the spreads about the peak centres, and σ_{X} and σ_{Y} are the standard deviation of the distribution. The standard deviations σ_{X} and σ_{Y} are indicative of the location accuracy. In practice, the axes of the elipse will rarely align perfectly with x and y axes, but instead will be rotated by an angle ⊖. To account for this, the general form of the Gaussian function fitted to the signal is expressed as:

$$f ( x , y ) = A exp ( - ( {a ( x - {x}_{o} )}^{2} + 2 b ( x - {x}_{o} ) ( y - {y}_{o} ) + {c ( y - {y}_{o} )}^{2} ) )$$

in which:

$$a = \frac{{cos}^{2} θ}{2 {σ}_{X}^{2}} + \frac{{sin}^{2} θ}{2 {σ}_{Y}^{2}}$$

$$b = − \frac{sin 2 θ}{4 {σ}_{X}^{2}} + \frac{sin 2 θ}{4 {σ}_{Y}^{2}}$$

$$c = \frac{{sin}^{2} θ}{2 {σ}_{X}^{2}} + \frac{{cos}^{2} θ}{2 {σ}_{Y}^{2}}$$

and the matrix:

$$[ \begin{matrix}a & b \\ b & c\end{matrix} ]$$

is positive-definite.

Optionally, the position data comprises only xo and yo for each fluorophore. Optionally, the position data also records σ_{X} and σ_{Y}. In instances where σ_{X} and σ_{Y} are significantly different for a given fluorophore, this may be indicative of two closely spaced fluorophores, and treated differently in the data analysis.

### Quantification

The methods of the invention allow more reliable quantitation/quantification of biomarker abundance compared to conventional methodologies, due to removing variability introduced by under-or over-counting.

Optionally, the number of target molecules is determined by carrying out iterations of the imaging cycle until the number of photoactivatable fluorophores activated during the photoactivation step falls below a cut-off value. The cut-off value may be, for example, fewer than 20 photoactivated fluorophores in the field of view, fewer than 10 photoactivated fluorophores in the field of view, fewer than 5 photoactivated fluorophores in the field of view, or no photoactivated fluorophores in the field of view. The total number of target molecules may be calculated by summing the number of detected target molecules across the full imaging procedure.

Alternatively, the number of target molecules may be determined by carrying out multiple iterations of the imaging cycle and then extrapolating the total number of target molecules. This extrapolation may be carried out by fitting a decrease in detected fluorophores over N_{cycle} imaging cycles, and then calculating the area under the curve. The fitting may be carried out, for example, using a non-linear regression model. The number of cycles, N_{cycle}, may be less than 200 cycles, less than 100 cycles, less 80 cycles, less than 60 cycles, less than 50 cycles, less than 40 cycles, less than 30 cycles or less than 20 cycles. The lower limit for N_{cycle} may be, for example, at least 10 cycles, or at least 20 cycles.

In situations where the target molecules are associated with an object, the method may comprise a step of associating each of the detected target molecules with an object, and determining the number of target molecules associated with that object.

The object may be, for example, a biological cell or part (e.g. substructure) thereof, a vesicle, a particle (such as a virion), or an aggregate (such as an amyloid plaque). Objects forming part of a biological cell may be, for example, the cell membrane, the nucleus, the nuclear membrane, the cytoplasm.

The cell may be an animal cell, bacterial cell or plant cell. Optionally, the cell is a human cell.

The step of associating each of the detected target molecules with an object may comprise clustering the detected positions of the fluorophores. This clustering may be carried out, for example, using Hierarchical Density-Based Spatial Clustering of Applications with Noise (HDBSCAN) analysis, as described, for example, in the publication “Density-Based Clustering Based on Hierarchical Density Estimates” by R. Campello, D Moulavi and J. Sander (Advances in Knowledge Discovery and Data Mining, pp 160-172, April 2013), as well as on the following web page: https://hdbscan.readthedocs.io/en/latest/how_hdbscan_works.html. Alternatively, clustering may be carried out using the methods taught in the applicant's own earlier applications WO 2022/053624 (see claims 1 to 8 of that application, and the accompanying description) and WO 2022/167483.

Alternatively, the step of associating each of the detected target molecules with an object may comprise identifying object boundary information from a reference image, and comparing the object boundary information with the fluorophore position data. The method may comprise obtaining a reference image of the same field of view as that subjected to imaging step (b), either prior to or after carrying out imaging step (b). The reference image may be a white light image. Alternatively, the reference image may be a fluorescence image from a non-photoactivatable fluorophore present on the object (in which case, care should be taken to avoid the non-photoactivatable fluorophore complicating measurement of the photoactivatable fluorophore of the invention).

### Biological Sample

In the present invention, “biological sample” refers to a tissue, a liquid, a cell isolated from an individual, or a mixture thereof. For example, the biological sample may be a cell suspension.

Examples thereof can include, but are not particularly limited to, biopsied tumour, spinal fluid, pleural fluid, intra-abdominal fluid, lymph, skin sections, blood, urine, faeces, sputum, the respiratory organs, the intestinal tract, the genitourinary tract, saliva, milk, the digestive organs, and cells collected therefrom.

Preferred examples of the “biological sample” can include a portion of resected tissue obtained during surgery performed for the purpose or treating cancer, a portion of tissue collected by biopsy or the like from a subject suspected of having cancer, and cells derived from blood, pleural fluid, or intra-abdominal fluid. The biological sample may be obtained from cancerous tissue or may contain cancer cells.

A sample as described herein may refer to any type of sample comprising cells, whether from a biological sample obtained from a subject, or from a sample obtained from e.g. a cell line. In embodiments, the sample is a sample obtained from a subject, such as a human subject. The sample is preferably from a mammalian (such as e.g. a mammalian cell sample or a sample from a mammalian subject, such as a cat, dog, horse, donkey, sheep, pig, goat, cow, mouse, rat, rabbit or guinea pig), preferably from a human (such as e.g. a human cell sample or a sample from a human subject).

The samples used may comprise tumour cells (e.g. a tumour sample or sample comprising circulating tumour cells). Such as sample may be a “mixed sample”. A “mixed sample” refers to a sample that is assumed to comprise multiple cell types. Within the context of the present disclosure, a mixed sample is typically one that comprises tumour cells or is assumed (expected) to comprise tumour cells, and normal cells. Samples obtained from subjects, such as e.g. tumour samples, are typically mixed samples (unless they are subject to one or more purification and/or separation steps). Typically, the sample comprises tumour cells and at least one non-tumour cell type (and/or genetic material derived therefrom). A “tumour sample” refers to a sample derived from or obtained from a tumour. Such samples may comprise tumour cells and normal (non-tumour) cells. The normal cells may comprise immune cells (such as e.g. lymphocytes), and/or other normal (non-tumour) cells (e.g. stromal cells). The lymphocytes in such mixed samples may be referred to as “tumour-infiltrating lymphocytes” (TIL). A tumour may be a solid tumour or a non-solid or haematological tumour. A tumour sample may be a primary tumour sample, tumour-associated lymph node sample, or a sample from a metastatic site from the subject. A sample comprising tumour cells may be a bodily fluid sample. A mixed sample may have been subject to one or more processing steps that may modify the proportion of the multiple cell types in the sample. For example, a mixed sample comprising tumour cells may have been processed to enrich the sample in tumour cells. Thus, a sample of purified tumour cells may be referred to as a “mixed sample” on the basis that small amounts of other types of cells may be present, even if the sample may be assumed, for a particular purpose, to be pure (i.e. to have a tumour fraction of 1 or 100%).

### Sample Preparation

The biological sample may be purified prior to labelling step (a), for example, by filtering or centrifugation. Preferably, this purification occurs before labelling, to avoid unwanted components of the sample scavenging the fluorescent probes.

In instances where the biological sample comprises cells, the sample is preferably fixed prior to imaging step (b). Fixation may be carried out prior to labelling step (a). Alternatively, fixation may be carried out after labelling step. Fixation may be implemented to eliminate Brownian motion of molecules during imaging, which otherwise complicate accurate localisation of fluorophores.

Fixation can be performed with any suitable fixative agents. Suitable fixative agents include, for example, formaldehyde, glutaraldehyde, or glyoxal.

A preferred fixative agent is paraformaldehyde, for example, 2% paraformaldehyde in phosphate buffered saline (PBS).

Optionally, any fixation step is followed by a quenching step, in which the biological sample is treated with a quenching agent. This helps to quench any autofluorescent background generated by the fixative agent, and also serves to inactivate unreacted fixative which might otherwise interfere/react with the probes. Suitable quenching agents include, for example, glycine (e.g. 1 mM glycine), sodium borohydride and ammonium chloride. Fixation may be carried out before or after labelling of the biological sample. Preferably, there is a fixation step after labelling, since this can help to retain the probes on the biological sample and so allows for sample storage and repeated analysis.

In implementations where the biological sample comprises objects, the method preferably involves the step of immobilising the objects on the imaging substrate. To promote immobilisation, the imaging substrate may be treated with a binding agent. The binding agent may be, for example, a non-specific binding agent (for example, a positively charged polymer, such as poly-L-lysine (PLL), poly-D-lysine (PDL), chitosan, or nitrocellulose) or a specific binding agent (for example, an antibody, antibody derivative or aptamer).

Preferably, the imaging substrate is treated with a passivation agent. Passivation of the imagine substrate helps to reduce nonspecific binding of impurities and reagents to the surface of the substrate. The passivation agent may be, for example, a protein (e.g. bovine serum albumin (BSA) or human serum albumin (HSA)), a nonionic surfactant (such as polysorbate 20 (e.g. Tween®-20), Triton X-100, or poloxamer 407 (e.g. Pluronic™ F127)), polyethylene glycol (PEG) (optionally in the form of an ester), or any mixture thereof.

Optionally, the labelling step (a) is carried out after immobilising objects on the imaging substrate.

Alternatively, the labelling step (a) is carried out prior to immobilisation of objects on the imaging substrate. Where labelling is carried out prior to immobilisation, this can more readily allow removal of non-bound probe, e.g. by centrifugation.

### Multiplexing

Often it is useful to image multiple different target molecules on the same cell. The inventors have identified a range of possibilities for carrying out this type of experiment.

### Multiplexing Using Multiple Colours

In one implementation, the methods of the present invention may comprise labelling the biological sample with at least two probes, wherein each of said probes comprises at least one photoactivatable fluorophore, and wherein the fluorescence emission from each probe is distinguishable.

The fluorescence emission from each probe may be distinguishable because, for example, the probes bear a fluorophore showing different emission characteristics. In particular, the probes may be distinguishable because their emission spectra show minimal overlap.

For example, in some implementations at least two probes having a photoactivatable fluorophore having a different emission colour. Typically, the photoactivatable fluorophores will also be most efficiently excited at different wavelength, in which case the excitation light source may operate at multiple excitation wavelengths (e.g. through use of two different lasers).

For example, the method may involve the use of two or more of the photoactivatable organic dyes indicated above.

Alternatively, or additionally, at least one probe may bear both a photoactivatable fluorophore and a non-photoactivatable fluorophore, wherein the non-photoactivatable fluorophore shows minimal excitation by both the activation light source and excitation light source, and the non-photoactivatable fluorophore has a different emission colour to the photoactivatable fluorophore, and wherein the photoactivatable fluorophore serves as a Forster Resonance Energy Transfer (FRET) donor to the non-photoactivatable fluorophore when in its photoactivated state.

In instances where the method involves the use of probes having different emission characteristics, the fluorescence microscope is a multi-colour fluorescence microscope. In such instances, the detection optics are capable of detecting and distinguishing multiple colour channels.

To facilitate multi-colour detection, fluorescence emission in different colour channels/bands may be separated and directed to separate pre-determined detector areas.

In one implementation, this is achieved by splitting the emission in different colour channels to separate cameras. However, whilst this retains a large field of view, it results in a relatively bulky and expensive construction.

In another implementation, multi-colour detection is achieved on a single camera by configuring distinct portions of the detector to detect different colour channels. For example, for two-colour channel imaging the emission may be split so that one colour channel is directed to one half of the camera detector, and another camera channel is directed to the other half of the camera detector. For, three or four colour imaging, the camera detector may be split into quarters, in an analogous fashion. The skilled reader is aware of how to achieve this using suitable optical components, and commercially available splitters are available to achieve this configuration, such as the Dual-View™ and Quad-View™ systems from Optical Insights, LLC.

Optionally, the emission is spread across more than one colour channel. In such instances, it is possible to distinguish between fluorophores using ratiometric imaging. In other words, in instances in which emission from a given fluorescent probe is detectable across multiple colour channels, the characteristic ratio of fluorescence in the multiple colour channels may allow the fluorescent probe to be identified. To give an example of ratiometric imaging, consider probe A and B, wherein excitation at a specific wavelength causes probe A to produce fluorescence in colour channel X, and probe B to produce fluorescence in colour channels X and Y, according to an intensity ratio Z. With knowledge of the intensity detected in channel Y and ratio Z, the individual contributions of probes A and B to the signal in channel X can be calculated. This can allow the use of more probes, without increasing overall complexity of the imaging system.

Additionally or alternatively, multi-colour detection is facilitated through the use of a dispersive element as part of the detection optics, such as a prism or grating. The dispersive element can spectrally spread fluorescence emission such that different wavelengths illuminate different parts of a detector. In preferred implementations the dispersive element is a prism. The prism is preferably a compound prism, such as a doublet compound prism. Advantageously, prisms can provide a compact structure for achieving dispersion with a combination of lower photon loss and lower (or no) deviation of emission compared to gratings. Optionally, fluorescent signal is split into at least two detection paths—a first path lacking a dispersive element and a second path having a dispersive element, wherein the first path provides diffraction-limited spots for obtaining position data and the second path provides line spectra for identifying the specific type of fluorophore creating the diffraction-limited spots.

In implementations used fluorophores of different colour, delivery of excitation light of different colours may be carried out in sequence. In such instances, the excitation wavelengths are preferably introduced in the sequence of decreasing wavelength—i.e. with longer wavelengths used before shorter wavelengths. This sequence can help to prevent cross-talk between colour channels.

### Multiplexing Using Sequential Measurement

Additionally or alternatively, multiplexing may be achieved by carrying out multiple iterations of full process steps (a) and (b), using a different probe.

For example, in one implementation the invention provides a method of carrying out single-molecule imaging of a target molecule in a biological sample, the method comprising carrying out multiple repeats of steps (a) and (b)
a) labelling target molecules of the biological sample with a first probe, the first probe comprising at least one photoactivatable fluorophore, to provide labelled target molecules;
b) imaging the biological sample through carrying out multiple imaging cycles, each imaging cycle having:
   i. an activation step, comprising delivering activation light from an activation light source to photoactivate a subset of the photoactivatable fluorophores into photoactivated fluorophores;
   ii. an excitation step, comprising illuminating the photoactivated fluorophores with an excitation light source, and measuring the detected fluorescence to identify the individual photoactivated fluorophores;
   iii. a photobleaching step, comprising photobleaching the individual photoactivated fluorophores;
      wherein the imaging cycles are repeated until the number of photoactivated fluorophores created in activation step falls below a cut-off value; and
   wherein each repeat of steps (a) and (b) is carried out using a different probe, which probe labels a different target molecule.

Optionally, high intensity activation light may be provided between each repeat, to try to avoid photoactivatable fluorophores of earlier probes being visible in later repeats.

In this approach, each of the different probes used may bear the same fluorophore. Advantageously, this can allow information about multiple targets to be obtained without increasing the complexity of the imaging apparatus. For example, measurement of several different target molecules can be carried out in an instrument using only one type of fluorophore and one colour channel (e.g. one excitation light source, and simple detection optics).

Optionally, multiplexing may be achieved using oligonucleotide barcodes. In this case, the repeats are carried out using different oligonucleotide hybridisation probes. In such embodiments, each probe comprises an oligonucleotide tag which can be hybridised with a complementary oligonucleotide bearing the photoactivatable fluorophore.

In such methodologies, the invention provides a method of carrying out single-molecule imaging of a target molecule in a biological sample, the method comprising:
(I) labelling target molecules of the biological sample with multiple types of probe precursor, wherein each type of probe precursor labels a different target molecule, and each type of probe precursor has an associated oligonucleotide tag;
(II) carrying out multiple repeats of steps (a) and (b)
a) forming a first probe by hybridising the oligonucleotide tag of a first type of probe precursor with its complementary oligonucleotide, wherein the complementary oligonucleotide bears a photoactivatable fluorophore;
b) imaging the biological sample through carrying out multiple imaging cycles, each imaging cycle having:
   i. an activation step, comprising delivering activation light from an activation light source to photoactivate a subset of the photoactivatable fluorophores into photoactivated fluorophores;
   ii. an excitation step, comprising illuminating the photoactivated fluorophores with an excitation light source, and measuring the detected fluorescence to identify the individual photoactivated fluorophores;
   iii. a photobleaching step, comprising photobleaching the individual photoactivated fluorophores;
      wherein the imaging cycles are repeated until the number of photoactivated fluorophores created in activation step falls below a cut-off value; and
   wherein each repeat of steps (a) and (b) is carried out using a different complementary oligonucleotide to hybridise a different probe precursor.

The skilled reader will appreciate that multiplexing methodologies above may be used in tandem or in sequence to achieve further multiplexing. For example, the sequential measurement method may be combined with multiple colour methods.

### Methods of Diagnosis

The present invention is particularly suited for use in medical diagnosis and treatment settings.

In one aspect, the present invention provides a method of identifying the presence or severity of a disease in a patient, the method comprising:
selecting a biomarker of the disease to serve as a target molecule;
measuring the abundance of the biomarker in a biological sample obtained from the patient using a method of the invention (either the first, tenth or eleventh aspects of the invention); and
comparing the measured abundance of the biomarker against reference data.

In another aspect, the present invention provides a method of identifying the suitability of a specific medical treatment for treating a patient suffering from a disease, wherein the method involves:
selecting a biomarker indicative of suitability for the specific medical treatment to serve as a target molecule;
measuring the abundance of the biomarker in a biological sample obtained from the patient using a method of the invention (either the first, tenth or eleventh aspects of the invention); and
comparing the measured abundance of the biomarker against reference data.

In these aspects, the methods preferably comprise measuring the abundance of the biomarker at a cellular level—that is, identifying the copy number of the biomarker for individual cells.

To achieve accurate quantification, it is preferred that at least 80% of the biomarkers bear only one photoactivatable fluorophore, more preferably at least 90% of the biomarkers, more preferably at least 95% of the biomarkers, more preferably at least 98% of the biomarkers, most preferably all of the biomarkers. This degree of labelling may be achieved using the strategies discussed above.

### (A) Detection and Quantification of Targets for Antibody-based Immunotherapies

Monoclonal antibody therapies are commonly used in the treatment of various tissue-borne diseases. In cases of mAb therapies intended to induce target cell death through activation of antibody-dependent cellular cytotoxicity (ADCC), the biologically relevant density of target molecules can be extremely low due to the highly sensitive nature of ADCC-effective immune cells, such as NK cells. One such therapy is the anti-HER2 mAb Trastuzumab, which is known to be effective in patients with apparently HER2-negative tumours as scored by current methods (see Hurvitz). Many mAb therapies also function through the inhibition/disruption of signalling by their target protein-for example, mAbs against HER2 (e.g. Trastuzumab, Pertuzumab), VEGFR2 (e.g. Ramucirumab), EGFR (e.g. Panitumumab), PDGFR (e.g. Olaratumab). In such cases the precise density of the target in question is likely to be highly impactful on the efficacy of the treatment, as the strength and nature of target signalling is defined by the number of actively signalling molecules.

Thus, in one embodiment the present invention provides a method of identifying the suitability of an antibody therapy for treating a patient suffering from a disease, wherein the method involves:
selecting a biomarker targeted by the antibody used in the antibody therapy to serve as a target molecule;
measuring the abundance of the biomarker in a biological sample obtained from the patient using a method of the invention (either the first, tenth or eleventh aspects of the invention);
comparing the measured abundance of the biomarker against reference data, wherein the reference data comprises a threshold abundance indicative of suitability for treatment.

Optionally, the disease is cancer.

In one embodiment, the method is used to identify the suitability of anti-HER2 therapy for a patient suffering from breast cancer (e.g. metastatic breast cancer), and the biomarker is HER2. For example, the anti-HER2 therapy may be Trastuzumab or Pertuzumab. Optionally, the biomarker is labelled using a capture molecule corresponding to the antibody used in the antibody therapy.

In one embodiment, the method is used to identify the suitability of anti-VEGFR2 therapy for a patient suffering from gastric cancer, and the biomarker is VEGFR2. For example, the antibody therapy may be Ramucirumab.

In one embodiment, the method is used to identify the suitability of anti-EGFR therapy for a patient suffering from (metastatic) colorectal cancer, and the biomarker is EGFR. For example, the antibody therapy may be Panitumumab.

### (B) Detection and Quantification of Targets for Cellular Immunotherapies

Similar to ADCC-driven target killing in cases of mAb therapy, many cellular immunotherapies (e.g. CAR-T therapy) are sensitive to extremely low target densities, below the sensitivity thresholds of current tissue-probing methods. The quantification of absolute biomarker densities using methods of the invention can therefore provide access to clinically relevant data that are not accessible to less sensitive technologies.

For example, in one implementation the present invention provides a method of identifying the suitability of CAR-T therapy for treating a patient suffering from a disease, wherein the method involves:
selecting a biomarker targeted by the CAR-T therapy to serve as a target molecule;
measuring the abundance of the biomarker in a biological sample obtained from the patient using a method of the invention (either the first, tenth or eleventh aspects of the invention);
comparing the measured abundance of the biomarker against reference data, wherein the reference data comprises a threshold abundance indicative of suitability for treatment.

Optionally, the disease is cancer, in particular blood cancer.

Optionally, the biomarker is selected from CTLA-4, PD-1, PD-L1, CD19 and CSF1R.

### (C) Detection and Quantification of Markers of Dysfunction or Disease

The presence and abundance of many biomarkers are indicative of tissue dysfunction or disease. More sensitive and precise detection of these markers can improve both confidence in clinical classifications (through greater precision) and the severity of disease that can be detected (i.e. detecting conditions at an earlier stage of development due to increased sensitivity). Examples include the detection of oncoantigens (e.g. HER2, EGFR, MUC1), tumour-associated markers (e.g. CEA, tyrosinase, AFP etc.), markers of tissue dysfunction (e.g. stress markers, autoimmune/inflammatory markers etc.), or markers of infection (e.g. pathogen-derived molecules).

### Methods of Treatment

In some embodiments, the methods of the present invention can be used in methods of treatment. In particular, the methods of diagnosis disclosed herein can be used in the context of a method of treatment comprising the administration of one or more therapeutic agents, or in a method of selecting a patient suffering from a disease for treatment with a specific medical treatment or therapeutic agent.

Thus, in some embodiments, the present invention provides a method of treating a patient having a disease, the method comprising:
selecting a biomarker of the disease to serve as a target molecule;
measuring the abundance of the biomarker in a biological sample obtained from the patient using a method of the invention (either the first, tenth or eleventh aspects of the invention);
comparing the measured abundance of the biomarker against reference data, thereby identifying the presence or severity of the disease in the patient; and administering one or more therapeutic agents to the patient.

In some embodiments, the present invention provides a method of selecting a patient suffering from a disease for treatment with a specific medical treatment, wherein the method involves:
selecting a biomarker indicative of suitability for the specific medical treatment to serve as a target molecule;
   measuring the abundance of the biomarker in a biological sample obtained from the patient using a method of the invention (either the first, tenth or eleventh aspects of the invention);
   comparing the measured abundance of the biomarker against reference data; and
   treating the patient with said specific medical treatment.

Optionally, the disease is cancer. In some embodiments, the disease is breast cancer (e.g. metastatic breast cancer), gastric cancer or colorectal cancer (such as metastatic colorectal cancer). In some embodiments, the disease is breast cancer (e.g. metastatic breast cancer) and the biomarker is HER2. In some embodiments, the disease is gastric cancer and the biomarker is VEGFR2. In some embodiments, the disease is colorectal cancer (e.g. metastatic colorectal cancer) and the biomarker is EGFR.

In some embodiments, the therapeutic agent or the specific medical treatment is an antibody. In some embodiments, the therapeutic agent or the specific medical treatment is an anti-HER2 therapy such as Trastuzumab or Pertuzumab. In some embodiments, the therapeutic agent or the specific medical treatment is an anti-VEGFR2 therapy such as Ramucirumab. In some embodiments, the therapeutic agent or the specific medical treatment is an anti-EGFR therapy such as Panitumumab.

In some embodiments, the capture molecule corresponds to the antibody used in the antibody therapy.

In some embodiments, the disease is cancer and the therapeutic agent or the specific medical treatment is a CAR-T therapy. In some embodiments, the cancer is blood cancer and the therapeutic agent or the specific medical treatment is a CAR-T therapy.

### BRIEF DESCRIPTION OF THE FIGURES

Embodiments and experiments illustrating the principles of the invention will now be discussed with reference to the accompanying figures in which:

FIG. 1 is a schematic of photoactivated fluorophores being identified on a cell using prior art PALM methodology, in which photoblinking of the fluorophores complicates counting of the true number of molecules;

FIG. 2 is a workflow showing a method of quantitating target molecules according to a first aspect of the present invention;

FIGS. 3A-3C show a schematic of a microscope carrying out the steps of the first aspect of the invention during the phases represented in FIG. 2;

FIG. 4A is a schematic of the identified locations of target molecules obtained for three cells in suspension using a method according to the first aspect of the invention, in which clustering analysis is used to determine cell boundaries;

FIG. 4B is a schematic of the identified locations of target molecules obtained for three cells in a fixed tissue sample using a method according to the first aspect of the invention, in which a white light image is used to determine cell boundaries;

FIG. 5 is a schematic representation of an antibody labelled with multiple copies of a photoactivatable fluorophore, as known from the prior art;

FIG. 6 is a schematic representation of a probe suitable for use in the first aspect of the present invention, comprising an antibody labelled with two identical nanobodies, each nanobody bearing a single photoactivatable fluorophore;

FIG. 7 is a schematic representation of a probe suitable for use in the first aspect of the present invention, comprising an antibody labelled with two identical nanobody fusion proteins, each having a protein tag labelled with a single photoactivatable fluorophore;

FIG. 8 is a schematic representation of a similar probe to that in FIG. 8, differing in that the protein tag of one of the nanobodies has been replaced by a non-fluorescently-labelled alternative protein tag;

FIG. 9A shows a schematic representation of a probe similar to that in FIG. 8, but in which the two protein tags are connected by an enzymatically cleavable linker;

FIG. 9B shows the probe of FIG. 9A after treatment of the cleavable linker with a protease;

FIG. 10 is a workflow showing methods for obtaining probes labelled with only a single photoactivatable fluorophore;

FIG. 11 is a plot showing the number of detected PA-JF646 and PA-JF549 fluorophores detected across 39 imaging cycles according to a method of the first aspect of the invention; and

FIG. 12 shows a DNA origami grid structure having PA-JF646 fluorophores positioned at a spacing of 20 nm, with FIG. 12A providing a schematic representation of the grid, FIG. 12B showing positional data of the grid obtained using dSTORM, and FIG. 12C showing positional data of the grid obtained using the method of the first aspect of the invention.

### DETAILED DESCRIPTION OF THE INVENTION

Aspects and embodiments of the present invention will now be discussed with reference to the accompanying figures. Further aspects and embodiments will be apparent to those skilled in the art. All documents mentioned in this text are incorporated herein by reference.

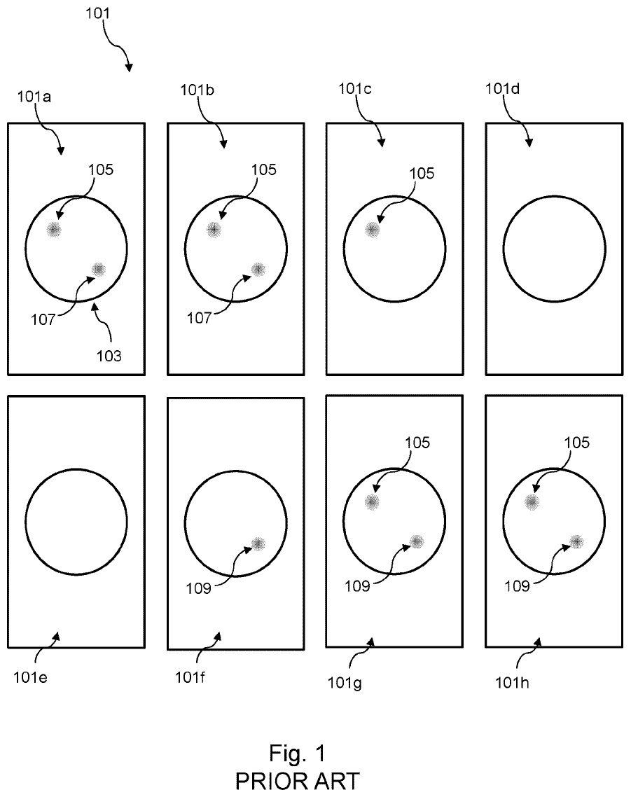

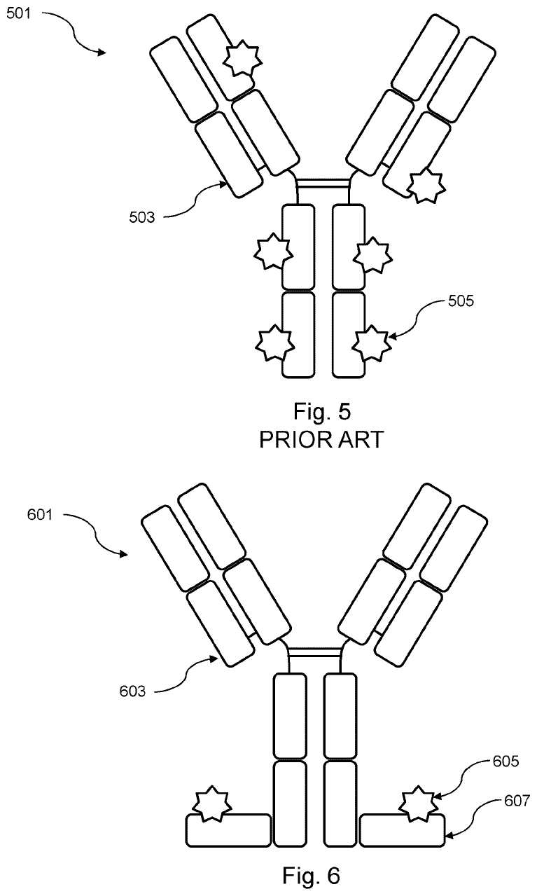

FIG. 1 represents an image stack 101, comprising eight video frames 101a-101h, showing the detected localisation of fluorescent molecules on a biological cell 103 using a prior art implementation of PALM. In this case, target molecules on the biological cell 103 are labelled with the monoclonal antibody depicted in FIG. 5, where the monoclonal antibody has been randomly labelled with several copies of a photoactivatable fluorophore, and where the degree of labelling on each monoclonal antibody shows variability. To carry out the measurement, UV activation light is applied continuously to randomly activate a subset of the photoactivatable fluorophores, and excitation light in the visible region is continuously supplied to excite any photoactivated fluorophores.

In frames 101a and 101b, two photoactivated fluorophores 105 and 107 are visible, each fluorophore being associated with an antibody bound to a target molecule. The shape depicted in FIG. 1 represents the detected fluorescence signal from the individual fluorophores, which can be approximated by a Gaussian function to localise the position of the fluorophore to sub-wavelength accuracy.

First photoactivated fluorophore 105 undergoes blinking in frames 101e and 101f, in which it temporarily switches into a dark state. The fluorescence returns in frames 101g and 101h.

Second photoactivated fluorophore 107 undergoes photobleaching prior to frame 101c, but a further photoactivatable fluorophore 109 associated with the same antibody as fluorophore 107 becomes photoactivated in frame 101g. Given the close spacing between fluorophore 107 and 109 on the antibody with which they are associated, the fluorophores appear in an identical position.

The time course of fluorescence presents challenges to identifying the true number of target molecules on the surface. The photoblinking of fluorophore 105 will lead to double-counting of fluorophore 105, unless the data analysis is designed to allow for photoblinking, e.g. by permitting three or fewer gap frames in this instance. However, if the data analysis software permits gap frames, then this could incorrectly assign the fluorescence from fluorophores 107 and 109 to a single fluorophore.

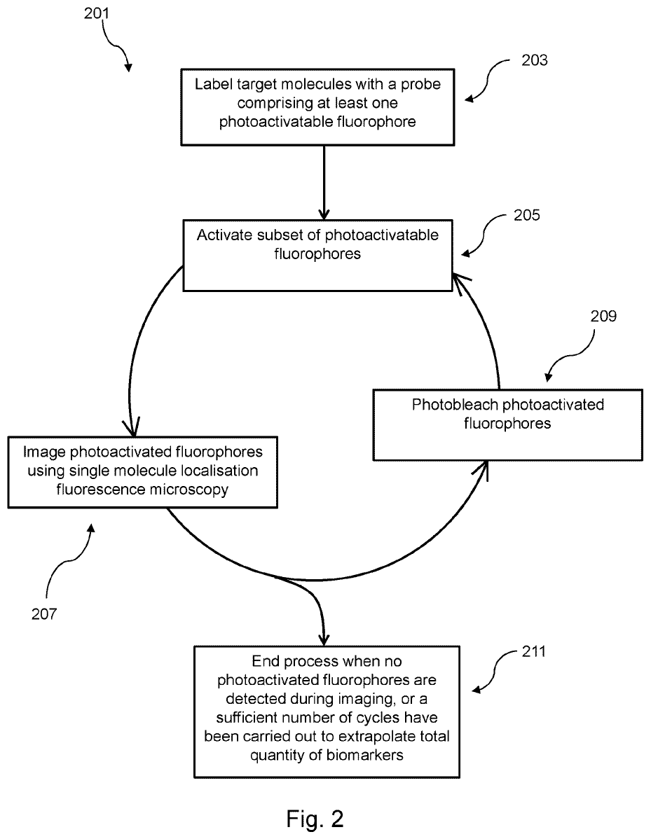

To address these issues, the inventors have developed the method 201 set out in FIG. 2.

The method begins with labelling step 203, in which target molecules in a sample are labelled with probes comprising at least one photoactivatable fluorophore. The method then moves to an iterative imaging step.

The imaging step begins with an activation step 205, in which a pulse of activation light is delivered to the sample to randomly photoactivate a subset of the photoactivatable fluorophores.

After delivery of the pulse of activation light, excitation light is supplied in excitation step 207, in order to image photoactivated fluorophores.

After a set time period, in photobleaching step 209 the intensity of the excitation light is increased in order to photobleach any photoactivated fluorophores in the sample, before returning to a further iteration of activation step 205.

These iterations are continued either until no photoactivated fluorophores are detected, or until a sufficient number of cycles have been carried out to extrapolate the total number of fluorophores, at which point the process ends at step 211.

Returning to FIG. 1, this method will avoid complexities during data analysis. In particular, the lack of activation light during the excitation step 207 means that if photoblinking occurs as depicted for fluorophore 105, one can be confident that this is in fact photoblinking instead of a second fluorophore becoming visible. In addition, the lack of activation light during the excitation step 207 avoids the possibility of second fluorophore 109 becoming activated during imaging.

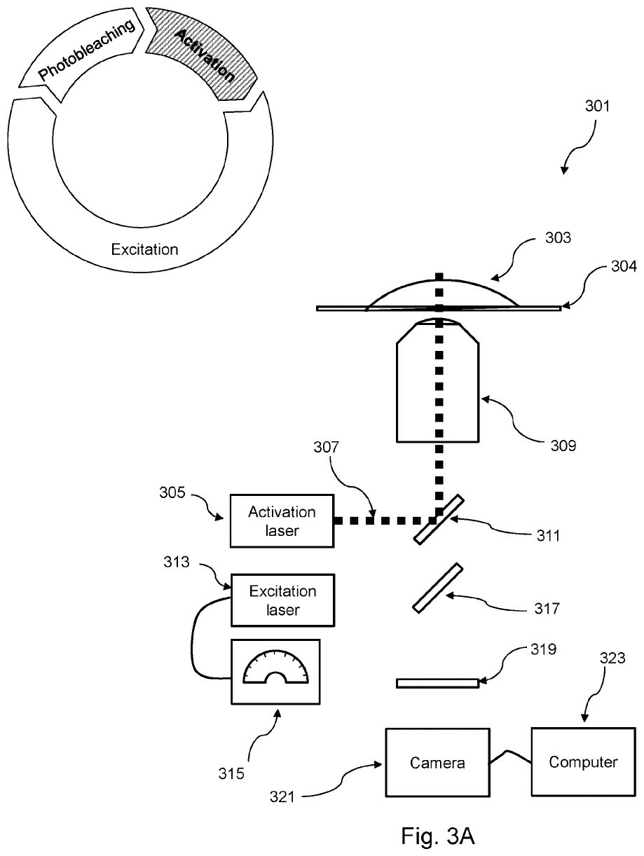

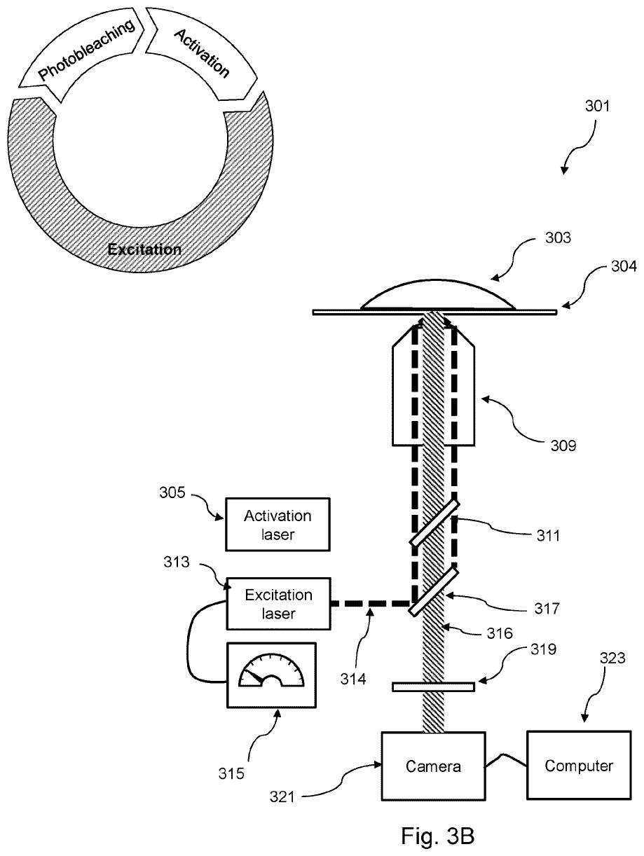

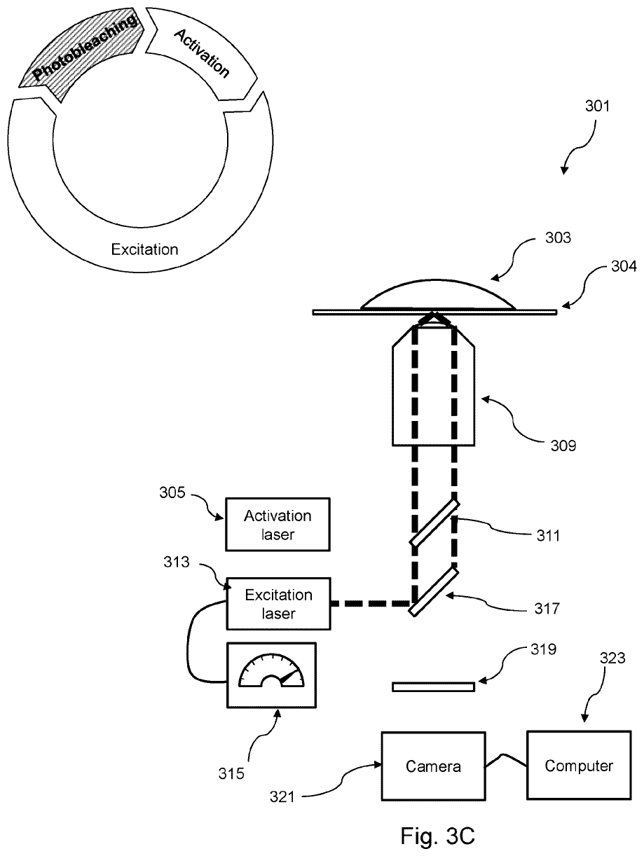

FIGS. 3A-C explain the method in more detail, with reference to an exemplary microscope 301 used during measurement of sample 303 held on microscope slide 304. In this case, sample 303 is a biopsy of diseased cells held in solution on microscope slide 304.

FIG. 3A depicts operation of microscope 301 during activation step 205. In this case, activation laser 305 delivers a pulse of UV light 307 at a wavelength of 405 nm via dichroic mirror 311 and objective lens 309. The light is delivered through the centre of objective 309 to give epifluorescence illumination, although other modalities are possible (such as TIRF).

In excitation step 207, the activation laser 305 is turned off, and excitation laser 313 is turned on to supply excitation light 314 at a low power setting using power supply 315. The excitation light 314 is delivered in an objective type TIRF modality, by directing excitation light 314 to the edge of objective lens 309 by dichroic mirror 317, such that it impinges on microscope slide 304 at an angle and undergoes total internal reflection at the interface between slide 304 and sample 303. However, as with the excitation light, the skilled reader recognises that other imaging modalities are possible, such as epifluorescence.

Fluorescence emission generated by excitation of the photoactivated fluorophores is collected by objective 309 and passes through dichroic mirrors 311 and 317, and through a filter 319 to remove unwanted interference from the excitation light 314 (in this case, the filter 319 is a bandpass filter, but other filters are possible, such as a longpass filter). The fluorescence is collected by camera 321, data from which is transferred to computer 323. The camera 321 is configured to collect data at a frame rate of 50 fps, corresponding to one frame every 20 ms.

After a certain excitation time has elapsed, the method moves to photobleaching step 209 in which the intensity of excitation laser 313 is increased by increasing the power supplied by power supply 315. In this case, the system is configured to implement the photobleaching step 209 after 50 frames have been recorded by the camera—i.e. after 1 second.

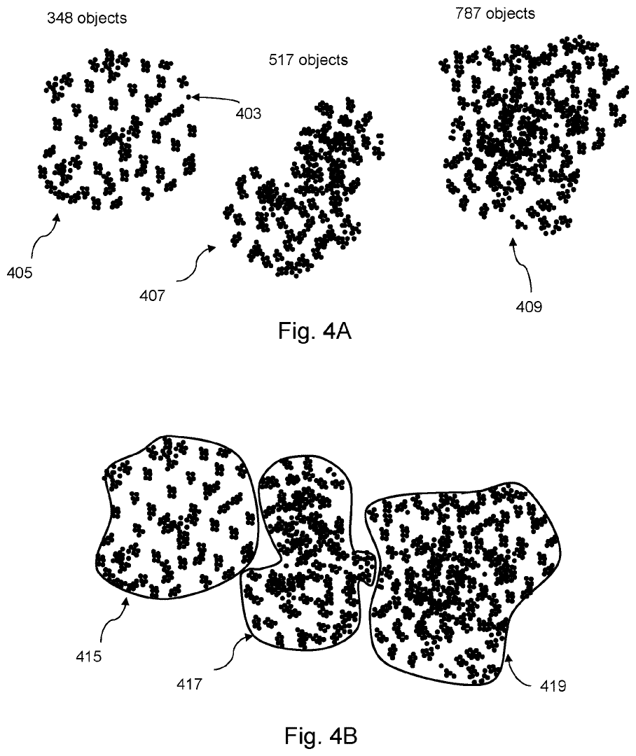

FIGS. 4A and 4B represent localisation data obtained according to the method of the invention, for a sample comprising fixed biological cells. In this instance, the target molecule is a membrane protein having a relatively low copy number per cell. The target molecule has been labelled using a probe as depicted in FIG. 8, having only a single photoactivatable fluorophore per probe.

FIG. 4A shows the locations of photoactivated fluorophores obtained by imaging according to the invention, with the locations accumulated across multiple imaging cycles until no further fluorescent signal is detected. Each circle 403 represents an individual detected molecule, with the centre of the circle positioned at the central point of the detected signal intensity when fitted with a Gaussian function, and the radius of the circle set to the standard deviation of the Gaussian fit. In this case, clustering analysis has been carried out, and has identified that the fluorophores are associated with three different cells 405, 407 and 409. Based on the clustering analysis, a putative cell boundary is determined, together with a calculation of the detected surface area within the boundary. Having clustered the data, the analysis counts the number of localisations associated with each cell, and determines that there are 348 localisations for cell 405, 517 localisations for cell 407 and 787 localisations for cell 409. The analysis then determines a density of the target molecules, based on the detected boundaries. Given that the probe used for labelling bears only a single photoactivatable fluorophore, both the copy number and the density give an accurate and unambiguous determination of the abundance of the protein on the cell surface.

In this case, the method also involves determining the level of clustering of the individual molecules using further analysis, such as that described in WO 2022/053624.

FIG. 4B shows data obtained from a tissue section, in which the cells 415, 417 and 419 appear in close proximity. In this instance, the proximity of the cells complicates accurate assignment of localisations to specific cells based on detected positions alone. Accordingly, the cell boundary is determined by white light illumination, which is overlaid on the localisation data. The cell boundaries are depicted in the figure as solid lines encompassing the localisation data.

FIGS. 5-9B depict probes suitable for use in the present invention.

FIG. 5 shows a prior art probe 501, comprising a monoclonal antibody 503 labelled with multiple copies of photoactivatable fluorophore 505. The photoactivatable fluorophore is an organic dye molecule. To achieve labelling, the monoclonal antibody 501 has been incubated with an excess of an amine-reactive form of the fluorophore, to randomly label primary amines on the monoclonal antibody. Monoclonal antibody 501 has several suitable primary amine sites, and in this case has resulted in five copies of the fluorophore 505 becoming attached. However, the sample of probes 501 has a distribution of labelling levels, between 1 and 6. This has led to a degree of labelling of 4.3. Accordingly, whilst the probe is compatible with the quantification methods of the invention, the multiple labels diminish the accuracy of quantification.

FIG. 6 depicts a probe 601 according to the present invention, comprising a monoclonal IgG antibody 603 having a nanobody 607 attached to each of its C_{H}3 domains. In this case, the nanobody 607 has been genetically engineered to contain only a single ectopic cysteine residue, which is subsequently labelled with a thiol-reactive photoactivatable organic dye 605. This labelling methodology ensures a reliable degree of labelling of two for probe 601. Accordingly, when wishing to count the number of target molecules, an accurate value can be obtained by taking the total number of detected fluorophores and dividing by two.

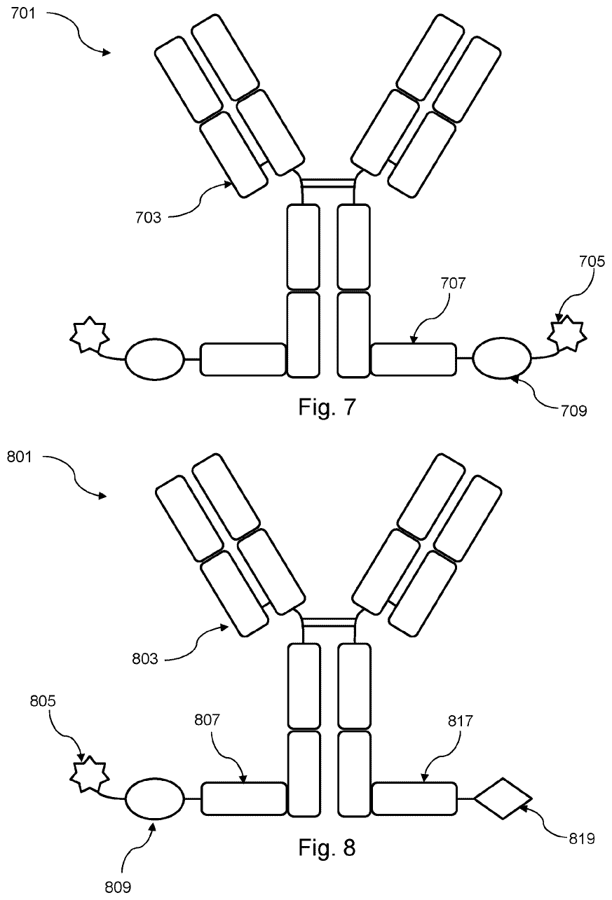

FIG. 7 depicts an alternative probe 701 according to the invention. In this case, instead of being genetically engineered to contain an ectopic cysteine, the nanobody 707 has been engineered to have a protein tag 709 fused at its terminus. In this case, the tag is a HaloTag®. The HaloTag® has been reacted with a haloalkane containing a single photoactivatable organic dye 705, so as to have only a single fluorophore per nanobody. When bound to antibody 703, this ensures that probe 701 has a degree of labelling of two. Advantageously, attaching the fluorophore to a protein tag domain, instead of to a residue in the nanobody itself, helps to increase the distance between the two fluorophores of probe 701. This distance helps to decrease unwanted interactions between the probes, such as the risk of resonance energy transfer.

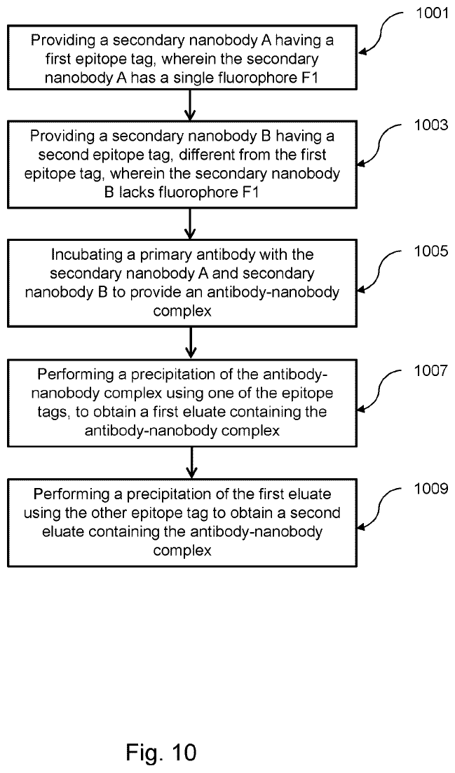

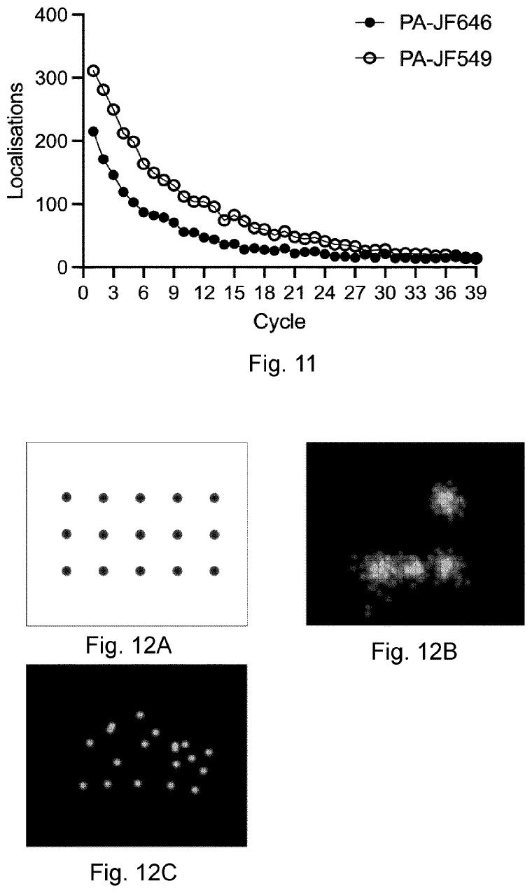

FIG. 8 shows a particularly preferable probe 801 according to the invention, bearing only a single photoactivatable organic dye 805. To achieve a degree of labelling of only one, the antibody 803 has been incubated with an equimolar mixture of (i) nanobody 807 having a protein tag 809 bearing photoactivatable organic dye 805, and (ii) nanobody 817 having a protein tag 819 lacking a photoactivatable fluorophore, where protein tag 809 and protein tag 819 are different. To select for single-labelled probe 801, the probe is labelled using the procedure set out in FIG. 10. Steps 1001 and 1003 involve providing the nanobodies 807 and 817, which are then used in incubation step 1005 to label the primary antibody 803 to form an antibody-nanobody complex. The resulting product will contain a mixture of probes bearing two copies of nanobody 807 (thereby having two fluorophores), two copies of nanobody 817 (thereby having no fluorophores), or a single copy each of nanobody 807 and nanobody 817 (thereby having one fluorophore). To select for the probes having a copy of each nanobody, a first eluate is obtained by carrying out a first immunoprecipitation directed to protein tag 809, and that eluate is then subjected to a second immunoprecipitation directed to protein tag 819. The skilled reader will recognise that the same effect could be achieved by first immunoprecipitating based on tag 819, before immunoprecipitating based on tag 809. Whilst FIG. 10 has been explained here in the context of protein tags, the skilled reader will recognise that the methodology will work for any type of epitope tag. Similarly, although the methodology has been explained using nanobodies as the secondary antibody fragment, the application envisages the use of other secondary antibody fragments, such as Fab, Fab′ and scFv.

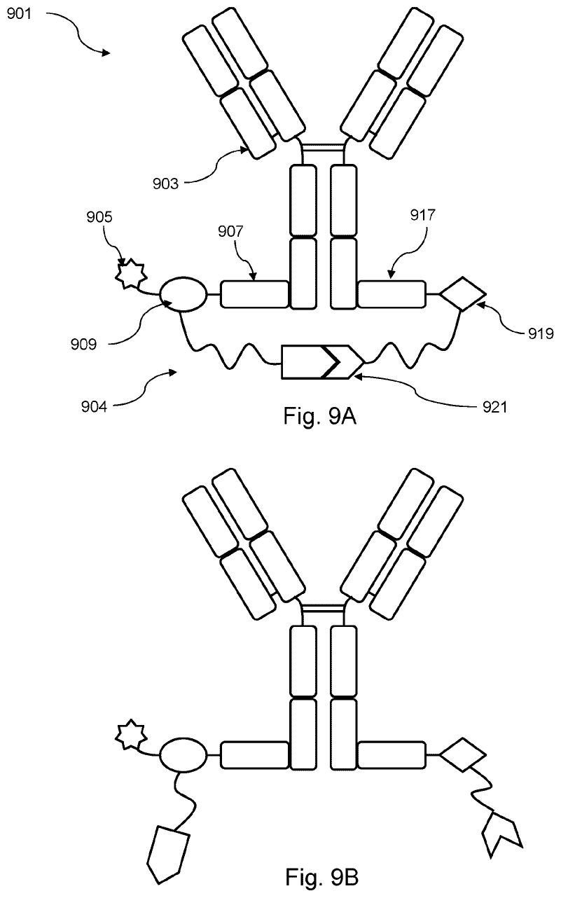

Turning now to FIGS. 9A and 9B, we see an alternative route which can be used to boost the yield of singly-labelled probes. In this route, probe 901 is formed by incubating antibody 903 with a construct 904. The construct 904 consists of nanobodies 907 and 917, each bearing a protein tag 909 and 919 respectively, which protein tags are linked by a cleavable linker 921. The nanobodies 907 and 917 are identical. The protein tag 909 bears a single copy of a photoactivatable organic dye 905. Thus, the probe 901 is identical to that depicted in FIG. 8, except for the presence of cleavable linker 921. In this instance, the cleavable linker 921 is a peptide linker, and the whole of construct 904 is a single fusion protein, which has been labelled with single photoactivatable organic dye 905. In this way, production of construct 904 can be simplified. Having nanobodies 907 and 917 present in the same construct increases the proportion of antibodies labelled with both nanobody 907 and 917 since after binding one nanobody intramolecular binding of the other nanobody from the same construct 904 is faster than binding by another construct.

After incubation, cleavable linker 921 is enzymatically cleaved. This breaks apart any cross-linked antibodies ahead of the purification steps 1007 and 1009 from FIG. 10.

### EXAMPLES

### Example 1—Photoactivatable Fluorophores on Glass

In a first set of experiments, a microscope slide was treated with an aqueous solution containing PA Janelia Fluor® 549, SE (PA-JF549) and PA Janelia Fluor® 646, SE (PA-JF646) at sufficiently low concentration to ensure sparse adsorption of molecules across the slide surface. Imaging was carried out according to the first aspect, with 39 cycles of the following illumination sequence at a camera rate of 33 frames per second (30 ms per frame):
1 frame of 405 nm illumination at a power of 1 mW
10 frames of 640 nm illumination at a power of 50 mW
10 frames of 560 nm illumination at a power of 50 mW
100 frames of 540 and 640 nm illumination at a power of 200 mW.

The number of detected photoactivated fluorophores across the field of view in each imaging cycle were then plotted, as shown in FIG. 12. The data show that the number of detected fluorophores decays with each cycle, as photoactivated fluorophores are bleached. In this instance, the predictable and rapid decay in detected fluorophores shows that an accurate estimate of the total number of fluorophores in the sample can be obtained after only a small number of imaging cycles.

The example also shows that multi-colour imaging can be carried out using the method of the invention whilst avoiding cross-bleaching that can occur in dSTORM, in part because excitation is carried out for only a short period at a relatively low laser power.

### Example 2—Quantification and Spatial Information

In a second set of experiments, a DNA origami structure was adsorbed on a microscope slide. Each structure consisted of PA-JF646-conjugated DNA which assembled into a 3×5 grid with 20 nm spacing, with fluorophores positioned at each branch point.

FIG. 12A is a schematic drawing of a 120 nm×100 nm area showing the theoretical position of each fluorophore when the grid is positioned perfectly flat and evenly spaced on the microscope slide.

FIG. 12B shows representative data obtained from the sample by dSTORM, with each spot representing the central location of a detected fluorophore and the diameter of the spot being indicative of the positional accuracy. The figure shows that many of the fluorophores have been missed due to bleaching or blinking, and those fluorophores which are observed are detected multiple times so as to give the appearance of clustered datapoints.

FIG. 12C shows data obtained using the PALM methodology according to the first aspect of the invention. The grid structure is far more visible—for example, the bottom row shows 5 fluorophores. The overall number of detected fluorophores is more representative of the total number of fluorophores present in the grid structure than the dSTORM data shown in FIG. 12B.

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

For the avoidance of any doubt, any theoretical explanations provided herein are provided for the purposes of improving the understanding of a reader. The inventors do not wish to be bound by any of these theoretical explanations.

Any section headings used herein are for organizational purposes only and are not to be construed as limiting the subject matter described.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means for example +/−10%.

### References

A number of publications are cited above in order to more fully describe and disclose the invention and the state of the art to which the invention pertains. Full citations for these references are provided below. The entirety of each of these references is incorporated herein.
Campello et al. “Density-Based Clustering Based on Hierarchical Density Estimates” Advances in Knowledge Discovery and Data Mining, pp 160-172, April 2013
Cnossen, J., et al. “Drift correction in localization microscopy using entropy minimization”, Optics Express, 2021, 29(8), pp 27961-27974
Grimm et al. “Bright photoactivatable fluorophores for single-molecule imaging” Nat Methods 13, 985-988 (2016)
Huang et al. “Three-dimensional Super-resolution Imaging by Stochastic Optical Reconstruction Microscopy”, Science, 2008, 319(5864), pp 810-813
Hurvitz, S. A. “DESTINY-Changing Results for Advanced Cancer”, N Engl J Med 2022; 387:75-76
Wiener et al. “Preparation of single-and double-oligonucleotide antibody conjugates and their application for protein analytics”, Sci Rep 10, 1457 (2020)
WO 2022/053624
WO 2022/167483
For standard molecular biology techniques, see Sambrook, J., Russel, D. W. Molecular Cloning, A Laboratory Manual. 3 ed. 2001, Cold Spring Harbor, New York: Cold Spring Harbor Laboratory Press

1. A method of carrying out single-molecule imaging of a target molecule in a biological sample, the method comprising:
a) labelling target molecules of the biological sample with a probe, the probe comprising at least one photoactivatable fluorophore;
b) imaging the target molecules through carrying out multiple imaging cycles, each imaging cycle having:
i. an activation step, comprising delivering activation light from an activation light source to photoactivate a subset of the photoactivatable fluorophores into photoactivated fluorophores;
ii. an excitation step, comprising illuminating the photoactivated fluorophores with an excitation light source, and measuring the detected fluorescence to identify the individual photoactivated fluorophores; and optionally
iii. a photobleaching step, comprising photobleaching the individual photoactivated fluorophores by illuminating the sample at a higher intensity than that used for the excitation step.

2. A method according to claim 1, wherein the method is for quantifying the presence of the target molecule.

3. A method according to claim 1, wherein the biological sample comprises cells.

4. A method according to claim 3, wherein the method is for quantifying the presence of the target molecule on individual cells.

5. A method according to claim 1, comprising said photobleaching step.

6. A method according to claim 5, wherein the photobleaching step involves illuminating the sample at a higher intensity than that used for the excitation step.

7. A method according to claim 6, involving illuminating using the excitation light source at a lower intensity during the excitation step, and illuminating using the excitation light source at a higher intensity during the photobleaching step.

8. A method according to claim 7, wherein the illumination intensity during the photobleaching step is at least 100% higher than the illumination intensity during the excitation step.

9. A method according to claim 1, further comprising monitoring the activation rate of the photoactivatable fluorophores and adjusting the operation of the activation light such that the activation rate falls within a desired threshold.

10. A method according to claim 1 [any one of the preceding claims], wherein the or each of the at least one photoactivatable fluorophore(s) is a photoactivatable organic dye.

11. A method according to claim 1 [any one of the preceding claims], wherein the degree of labelling of the target molecule with the photoactivatable fluorophore is between 1.0 and 2.0.

12. A method according to claim 11, wherein at least 80% of target molecules bear only one photoactivatable fluorophore.

13. A method according to claim 1 [any one of the preceding claims], wherein the probe is a capture molecule.

14. A method according to claim 13, wherein the probe is a primary antibody labelled with a secondary antibody or antibody fragment, wherein the secondary antibody or antibody fragment bears a controlled number of fluorophores.

15. A method according to claim 14, wherein the secondary antibody or antibody fragment is a monovalent antibody fragment or variant.

16. A method according to claim 15, wherein the monovalent antibody fragment or variant is a Fab, Fab′, single chain variable fragment (scFv) or nanobody.

17. A method according to claim 16, wherein the monovalent antibody fragment is a nanobody.

18. A method according to claim 17, wherein the nanobody has only 1 or 2 cysteine residues, and the controlled number of fluorophores corresponds to photoactivatable fluorophores attached to said 1 or 2 cysteine residues.

19. A method according to claim 17, wherein the nanobody has one or more tags, and the controlled number of fluorophores corresponds to photoactivatable fluorophores attached to said one or more tags.

20. A method according to claim 19, wherein the one or more tags are protein tags selected from HaloTag, SNAP-tag, or CLIP-tag.

21. A method according to claim 15, wherein the monovalent antibody fragment binds to an epitope on the constant region of the primary antibody.

22. A method according to claim 14, wherein the probe comprises only 1 fluorophore.

23. A method according to claim 22, wherein the probe comprises a primary antibody labelled with two monovalent antibody fragments, and wherein one of said monovalent antibody fragments bears a single copy of said photoactivatable fluorophore, and the other one of said monovalent antibody fragments bears no copies of said photoactivatable fluorophore.

24.A method according to claim 23, wherein the monovalent antibody fragments bind to an epitope on the constant region of the primary antibody.

25. A method according to claim 23, wherein the two monovalent antibody fragments are nanobodies.

26. A method of carrying out photoactivation localisation microscopy of photoactivatable fluorophores in a sample, comprising delivering activation light from an activation light source to photoactivate a subset of the photoactivatable fluorophores into photoactivated fluorophores, and imaging the photoactivated fluorophores, wherein the method comprises a feedback loop comprising monitoring the activation rate of the photoactivatable fluorophores and adjusting the operation of the activation light such that the activation rate falls within a desired threshold.

27. A method according to claim 26, wherein the activation rate is chosen so as to minimise the incidence of closely-spaced activated fluorophores observed during the excitation step.

28. A method according to claim 26, wherein the activation rate corresponds to the local density of fluorophores, calculated by assigning an area around the central position of a detected fluorophore in each frame, and calculating the number of fluorophores which occur within that area in the same frame.

29. A method according to claim 28, wherein the activation light source is configured so that the incidences of the activation rate being less than 0.2 fluorophores per μm² is less than 10%.

30. A method according to claim 28, wherein the incidences of the activation rate being more than 6 fluorophores per μm² is less than 10%.

31. A method according to claim 26 , wherein the feedback loop is computer-implemented.

32. A method of labelling a primary antibody with a single copy of a functional moiety F*, comprising:
a preparation step, comprising:
providing a secondary nanobody A having a first epitope tag, wherein the secondary nanobody A has a single copy of functional moiety F *;
providing a secondary nanobody B with a second epitope tag, different from the first epitope tag, wherein the secondary nanobody B lacks functional moiety F *;
wherein the secondary nanobody A or secondary nanobody B bind the same epitope on the primary antibody, or are cross-competing nanobodies;
an incubation step, comprising incubating the primary antibody with secondary nanobody A and secondary antibody B to provide an antibody-nanobody complex;
a purification step, comprising:
performing a precipitation of the antibody-nanobody complex using one of the epitope tags, to obtain a first eluate; and
performing a precipitation of the antibody-nanobody complex using the other epitope tag to obtain a second eluate;
wherein the second eluate comprises said primary antibody labelled with a single copy of functional moiety F *.

33. A fusion protein of formula:
(secondary nanobody A)-linker-(secondary nanobody B) wherein:
secondary nanobody A has a first epitope tag, and a single copy of functional moiety F* secondary nanobody B has a second epitope tag; and
linker is a cleavable linker.

34. A gene encoding the fusion protein of claim 33.

35. A gene construct comprising the gene of claim 34.

36. A cell comprising the gene construct of claim 35.

37. An antibody-nanobody complex comprising:
a primary antibody;
a secondary nanobody A, having a single fluorophore F1;
a secondary nanobody B, lacking a fluorophore F1;
wherein the secondary nanobody A and secondary nanobody B are bound to the primary antibody.

38. A composition comprising antibody-nanobody complexes, wherein at least 70% of the antibody-nanobody complexes present in the composition comprise:
a primary antibody;
a secondary nanobody A, having a single fluorophore F1;
a secondary nanobody B, lacking a fluorophore F1;
wherein the secondary nanobody A and secondary nanobody B are bound to the primary antibody.

39. Use of an antibody-nanobody complex of claim 37 in fluorescence microscopy.

40. A method of carrying out PALM imaging of a biological sample, the method comprising:
a) labelling target molecules of the biological sample with a probe, the probe being an antibody-nanobody complex according claim 37, wherein F1 is a photoactivatable fluorophore;
b) imaging the biological sample by carrying out PALM microscopy of fluorophore F1.

41. A method of carrying out PALM imaging of a biological sample, the method comprising:
a) labelling target molecules of the biological sample with a composition according to claim 38, wherein F1 is a photoactivatable fluorophore;
b) imaging the biological sample by carrying out PALM microscopy of fluorophore F1.

42. A method of identifying the presence or severity of a disease in a patient, the method comprising:
selecting a biomarker of the disease to serve as a target molecule;
measuring the abundance of the biomarker in a biological sample obtained from the patient using a method according to claim 1;
comparing the measured abundance of the biomarker against reference data.

43. A method of identifying the suitability of a specific medical treatment for treating a patient suffering from a disease, wherein the method involves:
selecting a biomarker indicative of suitability for the specific medical treatment to serve as a target molecule;
measuring the abundance of the biomarker in a biological sample obtained from the patient using a method of claim 1; and
comparing the measured abundance of the biomarker against reference data.

44. A method according to claim 42, wherein the disease is cancer.

45. A method according to claim 44, wherein the cancer is selected from breast cancer, gastric cancer or colorectal cancer.

46. A method according to claim 44, wherein the cancer is a blood cancer.

47. A method according to claim 43, wherein the specific medical treatment is an antibody.

48. A method according to claim 43, wherein the specific medical treatment is a CAR-T cell therapy.

49. A method according to claim 42, wherein the biomarker is selected from HER2, VEGFR2 or EGFR.

50. A method according to claim 42, wherein the biomarker is selected from CTLA-4, PD-1, PD-L1, CD19 and CSF1R.
